(19) United States
(12) Patent Application Publication
(10) Pub. No.: US 2023/0257707 A1
(43) Pub. Date: Aug. 17, 2023

(54) SYSTEMS AND METHODS FOR DIFFERENTIATING HEMATOPOIETIC CELLS

(71) Applicant: STEMCELL TECHNOLOGIES CANADA INC., Vancouver, CA (US)

(72) Inventors: Nooshin TABATABAEI-ZAVAREH, Vancouver (CA); Tim LE FEVRE, Vancouver (CA)

(21) Appl. No.: 18/017,512

(22) PCT Filed: Jul. 27, 2021
(86) PCT No.: PCT/CA2021/051050

§ 371 Date: Jan. 23, 2023

Related U.S. Application Data
(60) Provisional application No. 63/057,037, filed on Jul. 27, 2020.

(51) Int. Cl. C12N 5/0783; C12N 5/0781

(52) U.S. Cl. CPC C12N 5/0636; C12N 5/0646; C12N 5/0635; C12N 2501/26; C12N 2501/125; C12N 2501/145; C12N 2501/155; C12N 2501/115; C12N 2501/165; C12N 2500/90; C12N 2502/13; C12N 2506/02

(57) ABSTRACT

Disclosed are media, methods, systems and kits for differentiating hematopoietic progenitor cells from a population of early mesoderm cells under conditions that exclude any combination of certain exogenously added agonists of growth factor signaling. The population of early mesoderm cells may be derived from pluripotent stem cells, and the hematopoietic progenitor cells differentiated under the disclosed conditions are multipotent. The culture conditions disclosure herein may form serum-free workflows and/or workflows free of stroma and/or feeder cells.

### RELATED APPLICATION

This application claims the benefit of U.S. Provisional Patent Application No. 63/057,037, filed Jul. 27, 2020, the entire content of which is hereby incorporated by reference in its entirety.

### TECHNICAL FIELD

This disclosure relates to cell culture applications, and more particularly to cell culture applications with hematopoietic cells. Specifically, this disclosure relates to cell culture applications to differentiate hematopoietic cells.

### BACKGROUND

NK cells, B cells and T cells are lymphocytes that provide defense against pathogens and tumors. NK cells play a critical role in innate immunity, capable of secreting proinflammatory cytokines as well as killing cancerous or virus-infected cells. T cells are cells of the adaptive immune system. They recognize a wide range of targets through their antigen-specific T cell receptors (TCRs) and exert effector functions including cytokine secretion and cytotoxic cell killing. B cells are cells of the adaptive immune system. Among their various functions, certain B cells either produce or may be stimulated to produce antibodies.

Human NK, B and T cells can be generated by culturing CD34⁺ hematopoietic stem and progenitor cells (HSPCs) purified from cord blood (CB), bone marrow (BM) or peripheral blood (PB) with stromal cells and cytokines. However, cells derived from CB, BM, and PB are often heterogeneous and can vary in quality from donor to donor. Additionally, cells derived from CB, BM and PB are limited in number.

Pluripotent stem cells (PSCs) provide an opportunity to create homogenous, customizable, large-scale populations appropriate for clinical applications. Use of PSC also enables gene engineering methods that facilitate disease modeling or cell therapy applications.

Differentiating PSCs into NK, B and T cells will provide a useful tool for developing adoptive immunotherapy, for example, in cancer patients, as well as research into basic biology of these cells. Accordingly, there is a need for efficient NK cell, B cell and T cell differentiation protocols beginning from PSC.

### SUMMARY

The present disclosure relates to methods of differentiating hematopoietic cells.

In one aspect of this disclosure is provided a method of differentiating hematopoietic progenitor cells, comprising culturing a population of early mesoderm cells in a first medium, wherein the first medium comprises a basal medium supplemented with one or more of thrombopoeitin (TPO), stem cell factor (SCF) and FMS-like tyrosine kinase 3 ligand (FLT3L), and excluding any combination of exogenously added agonists selected from the list consisting of an agonist of BMP signaling, an agonist of fibroblast growth factor (FGF) signaling, and an agonist of vascular endothelial growth factor (VEGF) signaling.

In one embodiment, the agonist of BMP signaling is a bone morphogenic protein. In one embodiment, the bone morphogenic protein is BMP4.

In one embodiment, the agonist of FGF signaling is a fibroblast growth factor. In one embodiment, the fibroblast growth factor is FGF2.

In one embodiment, the agonist of VEGF signaling is vascular endothelial growth factor.

In one embodiment, the first medium is serum-free.

In one embodiment, the methods further comprise culturing the population of early mesoderm cells in the absence of exogenously added stroma- and/or feeder-cells. In one embodiment, culturing the population of early mesoderm cells in the first medium is for between 3 and 15 days.

In one embodiment, the hematopoietic progenitor cells are CD34+. In one embodiment, the hematopoietic progenitor cells have myeloid, erythroid, and lymphoid potential.

In one embodiment, the population of early mesoderm cells are derived from pluripotent stem cells (PSC).

In one embodiment, the methods further comprise culturing the PSC in a derivation medium, wherein the derivation medium comprises a second basal medium supplemented with one or more of the agonist of BMP signaling, the agonist of FGF signaling, and the agonist of VEGF signaling to obtain a differentiated population of PSC-derived early mesoderm cells.

In one embodiment, the agonist of BMP signaling in the derivation medium is a bone morphogenic protein. In one embodiment, the bone morphogenic protein is BMP4.

In one embodiment, the agonist of FGF signaling in the derivation medium is a fibroblast growth factor. In one embodiment, the fibroblast growth factor is FGF2.

In one embodiment, the agonist of VEGF signaling in the derivation medium is vascular endothelial growth factor.

In one embodiment, the derivation medium is serum-free.

In one embodiment, the methods further comprise culturing the PSC in the absence of exogenously added stroma- and/or feeder cells. In one embodiment, culturing the PSC in the derivation medium is for between 1 and 7 days.

In one embodiment, the methods further comprise forming the PSC into aggregates prior to culturing the PSC in the derivation medium. In one embodiment, the methods further comprise forming the PSC into aggregates in the derivation medium. In one embodiment, the aggregates are formed in a microwell device.

In one embodiment, the methods further comprise obtaining lymphoid progenitor cells after culturing the differentiated hematopoietic progenitor cells in a lymphoid differentiation medium under serum-free conditions and/or in the absence of exogenously added stroma- and/or feeder cells.

In one embodiment, the lymphoid progenitor cells have multi-lineage potential.

In one embodiment, the lymphoid progenitor cells have T cells and NK cells potential.

In one embodiment, the T cells and NK cells are functional.

In another aspect of this disclosure are provided media for differentiating hematopoietic progenitor cells from a population of early mesoderm cells, the media comprising a basal medium supplemented with one or more of thrombopoeitin (TPO), stem cell factor (SCF) and FMS-like tyrosine kinase 3 ligand (FLT3L), and excluding any combination of exogenously added agonists selected from the list consisting of an agonist of BMP signaling, an agonist of fibroblast growth factor (FGF) signaling, and an agonist of vascular endothelial growth factor (VEGF) signaling.

In one embodiment, the population of early mesoderm cells is derived from pluripotent stem cells. In one embodiment, the pluripotent stem cells are mammalian.

In one embodiment, the medium is serum-free. In one embodiment, the medium is effective in stroma- and/or feeder cell-free workflows.

In one embodiment, the agonist of fibroblast growth factor (FGF) signaling is a fibroblast growth factor. In one embodiment, the fibroblast growth factor is FGF2.

In one embodiment, the agonist of BMP signaling is a bone morphogenic protein. In one embodiment, the bone morphogenic protein is BMP4.

In one embodiment, the agonist of vascular endothelial growth factor (VEGF) signaling is VEGF.

In one embodiment, the hematopoietic progenitor cells have lymphoid, myeloid, and erythroid potency. In one embodiment, the hematopoietic progenitor cells are CD34+.

In another aspect of this disclosure are provided systems and/or kits including components (as described herein) for differentiating hematopoietic progenitor cells from pluripotent stem cells through a population of early mesoderm cells. The systems and/or kits may also include components for enriching any population of cells differentiated in a step-wise fashion (using the media and other culture coatings or cultureware disclosed herein). The systems and/or kits may also include components for differentiating hematopoietic progenitors to a population of multipotent lymphoid progenitors.

Other features and advantages of the presently disclosed subject matter will become apparent from the following detailed description. It should be understood, however, that the detailed description and the specific examples while indicating preferred embodiments of the disclosed subject matter are given by way of illustration only, since various changes and modifications within the spirit and scope of the invention will become apparent to those skilled in the art from this detailed description.

### BRIEF DESCRIPTION OF DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows bar graphs of output hematopoietic progenitor cells differentiated in the presence or absence of various growth factor combination. The frequency (FIG. 1A) and yield (FIG. 1B) of CD34⁺ hematopoietic progenitor cells per well of a 6-well Aggrewell™ plate were determined across WLS-1C, STiPS-M001, H1, and H9 PSC lines that were differentiated (in different combinations of BMP4, FGF, and VEGF agonsim) in accordance with the methods of this disclosure. FIG. 1C shows the frequency and yield of CD34⁺ hematopoietic progenitor cells per well of a 6-well Aggrewell™ plate were determined in the absence of only VEGF across WLS-1C, STiPS-M001, H1, and H9 PSC lines that were differentiated in accordance with the methods of this disclosure. FIG. 1D shows that increases in CD34⁺ cell differentiation (when differentiated in the absence of VEGF) carry through when querying the frequency and yield of CD5⁺CD7⁺ lymphoid progenitor cells. Data represents the mean of 2-5 experiments depending on the PSC cell line.

FIG. 2 shows a bar graph of the number of hematopoietic progenitors (A) and the percent viable hematopoietic progenitors (B) obtained using various aggregate dissociation reagents. Aggregates of 1,000 PSC were formed in each well of an Aggrewell™ 400 and total aggregates were pooled then split into 6 equals fractions, where they were dissociated using either 1-Accutase™2-TrypLE™ Express, 3-Collagenase II & TrypLE™ Express, 4-Collagenase IV & TrypLE™ Express, 5-Gentle Cell Dissociation Reagent (GCDR), or 6-Trypsin. Data represent the mean of 2-3 experiments as shown by individual dots.

FIG. 3 shows a bar graph of output hematopoietic progenitor cells differentiated from various PSC lines. The frequency and yield of CD34⁺ hematopoietic progenitor cells per well of a 6-well Aggrewell™ plate was determined across H1, H9, WLS-1C, STiPS-M001, and STiPS-F016 PSC lines that were differentiated in accordance with the methods of this disclosure (A). Data represent the mean±SEM of 7-22 experiments depending on the PSC cell line. Representative flow cytometry plots are shown for pre-enrichment (B) and post-enrichment (C) differentiated CD34⁺ hematopoietic progenitor cells.

FIG. 4 shows bar graphs of the multipotency of the CD34⁺ hematopoietic progenitor cells. The CD34+ progenitor cells differentiated in the absence of VEGF were cultured in MethoCult™ to assess their ability to differentiate to myeloid and erythroid lineages (A). Also, the CD34+ cells of this disclosure were further cultured in the StemSpan™ Lymphoid Expansion system at varying cell numbers to assess the progenitor cell frequency at limiting dilution. Each cell number was plated in twelve replicate wells, and the number of positive wells (wells containing lymphoid progenitor cells) for each cell number was used to determine the frequency. The data for each condition are summarized in the table (B).

FIG. 5 shows a bar graph of output lymphoid progenitor cells differentiated from hematopoietic progenitor cells. The frequency and yield per input CD34⁺ cell of CD5⁺CD7⁺ lymphoid progenitor cells were determined across H1, H9, WLS-1C, STiPS-M001, and STiPS-F016 PSC lines that were differentiated from PSC-derived hematopoietic progenitor cells in accordance with methods of this disclosure (A). Data represent the mean±SEM of 5-20 experiments depending on the PSC cell line. Representative flow cytometry plot of PSC-derived CD5⁺CD7⁺ lymphoid progenitor cells (B)

FIG. 6 shows bar graphs of output hematopoietic progenitor cells and lymphoid progenitor cells when varying the duration of an early step of the differentiation protocol. The frequency and yield per well of a 6-well Aggrewell™ plate of CD34⁺ hematopoietic progenitor cells were determined across 1C, H1 and H9 PSC lines that were differentiated for either 10 or 12 days in accordance with methods of this disclosure (A). The frequency and yield per input CD34⁺ cell of CD5⁺CD7⁺ lymphoid progenitor cells were determined across 1C, H1 and H9 PSC lines that were differentiated for either 10 or 12 days in accordance with methods of this disclosure (B). Data in (A) and (B) represent the mean of 3-7 experiments depending on the cell line.

FIG. 7 shows bar graphs of output CD4⁺CD8⁺ double-positive T cells differentiated from hematopoietic progenitor cells. The frequency and yield per input CD34⁺ cell of CD4⁺CD8⁺ double-positive T cells were determined across H1, H9, WLS-1C, STiPS-M001, and STiPS-F016 PSC lines that were differentiated from PSC-derived lymphoid progenitor cells in accordance with methods of this disclosure (A). The frequency of CD3⁺TCRαβ⁺ cells (gated on CD4⁺CD8⁺ double-positive T cells) was determined across H1, H9, WLS-1C, STiPS-M001, and STiPS-F016 PSC lines that were differentiated from PSC-derived lymphoid progenitor cells in accordance with methods of this disclosure (B). A representative flow cytometry plot of output CD4⁺CD8⁺ double-positive T cells (C). A representative flow cytometry plot of output of CD3⁺TCRαβ⁺ cells gated on CD4⁺CD8⁺ cells (D). Data in (A) and (B) represent the mean±SEM of 3-13 experiments depending on the PSC line.

FIG. 8 shows bar graphs of output CD4⁺CD8⁺ double-positive T cells differentiated from lymphoid progenitors. The effect of seeding sorted viable cells or CD7⁺ cells among the differentiated lymphoid progenitors on the frequency and yield per input CD34⁺ cell of CD4⁺CD8⁺ double-positive T cells were determined across H1, WLS-1C, and H9 PSC lines (A). The effect of varying the number of lymphoid progenitor cells seeded on the frequency and yield per input CD34⁺ cell of CD4⁺CD8⁺ double-positive T cells was determined in H1 (dot symbol) and WLS-1C (square symbol) PSC lines (B). Data in (A) and (B) represent the mean of 1-4 experiments depending on the PSC line.

FIG. 9 shows a bar graph of output CD8⁺ single positive (SP) T cells (CD3⁺TCRαβ⁺CD4-CD8⁺) differentiated from PSC-derived CD4⁺CD8⁺ double-positive T cells. The frequency of CD8⁺ single positive T cells as differentiated in accordance with methods of this disclosure from H1, H9, and WLS-1C PSC lines is shown (A). Data are represented as mean±SEM of 3-4 experiments depending on the PSC cell line. Representative flow cytometry plots of expression of different T cell markers by the output CD8⁺ single positive T cells (B)-(E).

FIG. 10 shows bar graphs of output CD56⁺ NK cells differentiated from lymphoid progenitors. The frequency and yield per input CD34⁺ cell of CD56⁺ NK cells were determined across H1, H9, WLS-1C, STiPS-M001, and STiPS-F016 PSC lines that were differentiated into lymphoid progenitor cells in accordance with methods of this disclosure (A). Data are represented as mean±SEM of 4-13 experiments depending on the PSC cell line. Representative flow cytometry plots of NK cell marker co-expression of output CD56⁺ NK cells derived from STiPS-M001 PSC (B)-(H).

FIG. 11 shows bar graphs and flow cytometry plots demonstrating that PSC-derived NK cells are functional. Differentiated CD56⁺ NK cells exhibit comparable killing capacity across multiple PSC lines as peripheral blood (PB) NK cells at 2.5:1 effector (NK cell) to target (K562 cell) ratio (A). Data are represented as mean±SEM of 3-7 experiments depending on the PSC cell line. Representative flow cytometry plots of CD107a and IFN-γ expression by H1-derived NK cells that were either not activated (B) or activated (C) by co-culture with K562 cells at a 1:1 NK cell: K562 cell ratio, in comparison to peripheral blood NK cells activated by co-culture with K562 cells (D). Expression of IFN-γ (E) and CD107a (F) by CD56⁺ NK cells differentiated from various PSC lines, in comparison to PB CD56⁺ NK cells that were activated by co-culture with K562 cells. Data are represented as mean±SEM of 2-4 experiments depending on the PSC cell line.

FIG. 12 shows bar graphs of output hematopoietic progenitor cells and lymphoid progenitor cells when starting PSCs were maintained in different media formulations. Yield of CD34⁺ hematopoietic progenitor cells per well of a 6-well Aggrewell™ plate was determined across H1, H9, STiPS-F016 and WLS-1C PSC lines that were maintained in either mTeSR1 or TeSR-E8 (A), or in either mTeSR1 or mTeSR Plus (C). Data in (A) and (C) represent the mean of four experiments using mTeSR1 and two experiments using TeSR-E8. The frequency and yield per input CD34⁺ cell of CD5⁺CD7⁺ lymphoid progenitors were determined in H1, H9, STiPS-F016 and WLS-1C PSC lines that were maintained in either mTeSR1 or TeSR-E8 (B), or in either mTeSR1 or mTeSR Plus (D). Data in (B) and (D) represent the mean of seven experiments using mTeSR1 and 2 experiments using mTeSR Plus.

### DETAILED DESCRIPTION

The description that follows relates to media, methods, and systems (kits) for differentiating cells of the hematopoietic lineage from pluripotent stem cells (PSC) or early mesoderm cells.

Where used herein the term “pluripotent stem cell” or “PSC” refers to a cell that is capable of self-renewal and/or differentiating to any cell type of any/all of the three embryonic germ layers. PSC, such as embryonic stem cells, may be isolated from a blastocyst and subjected to either maintenance or differentiation cell culture conditions. PSC, such as induced pluripotent stem cells, may be derived from any cell type by the forced expression of certain pluripotency genes, such as Oct4, Nanog, Sox2, Klf4, etc. The forced expression of pluripotency genes may be accomplished via introducing their coding regions, whether stably or transiently, into a host cell or by introducing factors that activate the expression of endogenous copies of these genes in a host cell.

Where used herein, the term “early mesoderm cells” or “mesoderm precursor cells” are precursors of hematopoietic stem and progenitor cells. They may be isolated from an appropriate tissue, such as embryos (e.g. aorta-gonad-mesonephros) or are differentiated from PSC. Additionally, early mesoderm cells may correspond to a specialized subset of vascular endothelium known as hemogenic endothelium. Regardless of whether or not the population of early mesoderm cells are PSC-derived or isolated from early stage embryos, they may express one or more of KDR, CD56 (NCAM), or Brachyury, but may lack expression of markers such as CD326 (EpCAM).

Where used herein the term “hematopoietic stem and progenitor cell” or “HSPC” refers to a cell of the hematopoietic lineage that is capable of self-renewal and/or differentiating into a more specialized cell of the hematopoietic lineage. HSPC may be obtained from bone marrow (BM), umbilical cord blood (CB), embryonic through to adult peripheral blood (PB), thymus, peripheral lymph nodes, gastrointestinal track, tonsils, gravid uterus, liver, spleen or any other tissue having localized populations of HSPC. HSPC may also be differentiated from pluripotent stem cells such as induced pluripotent stem cells, embryonic stem cells, naïve stem cells, extended stem cells, or the like. A hallmark of HSPC is the expression of the transmembrane phosphoglycoprotein CD34, thus HSPC may be referred to as CD34⁺ cells. Human HSPCs may be further defined by expression of CD45 and CD34, and may be still further defined by combinations of markers such as CD38, CD43, CD45RO, CD45RA, CD10, CD49f, CD59, CD90, CD109, CD117, CD133, CD166, HLA-DR, CD201, and integrin-alpha3 which may be used to distinguish HSPC subsets. HSPCs may lack expression, or have only low expression, of markers such as Glycophorin A, CD3, CD4, CD8, CD14, CD15, CD19, CD20 and CD56; such markers may be characteristic of more mature blood cells. The term “hematopoietic progenitor cell” may be used interchangeably with either “hematopoietic stem and progenitor cell” or “HSPC”.

Where used herein, the term “lymphoid progenitor” refers to a cell type that is more specialized than a HSPC but is capable of further differentiating into one or more lymphoid cell types, such as B cells, T cells, or NK cells. A lymphoid progenitor cell may be a direct descendant of a HSPC or may be further removed from a HSPC. Further, a lymphoid progenitor cell may directly differentiate into a downstream lymphoid cell type or may undergo one or more further steps of differentiation before becoming a lymphoid cell type. One example of a lymphoid progenitor cell is a cell that is positive for the phenotypic markers CD7 and CD5. In another example, a lymphoid progenitor cell may be positive for CD7 but negative for CD5, or vice versa. In another example, a lymphoid progenitor cell may be negative for both CD7 and CD5. Other phenotypic markers that may be expressed by lymphoid progenitor cells include CD10, CD45RA, CD34, CD38, CD161, CD122, CD117, CD127, CD1a and/or integrinB7. Herein, unless explicitly stated, a population of lymphoid progenitor cells may refer to a homogeneous population of cells or a heterogeneous population of cells capable of differentiating to one or more lymphoid cell types. In one embodiment, a lymphoid progenitor may be capable of differentiating into any type of lymphoid cells. In one embodiment, a lymphoid progenitor may be more restricted in its differentiation capacity, and may only differentiate into one or more, but not all, types of lymphoid cells.

Where used herein, the term “natural killer cell” or “NK cell” refers to a type of lymphocyte of the hematopoietic lineage that may derive from a HSPC. More specifically, NK cells may derive from multilymphoid progenitors (MLPs) or common lymphoid progenitors (CLPs). NK cells are typically characterized by: the absence of T and B cell-specific markers; the expression of CD56 with or without CD16 (low affinity Fc gamma receptor 3A, expressed on a subset of NK cells); and their effector functions. More specifically, effector functions of NK cells may include cytotoxicity and/or the production of inflammatory cytokines such as IFNγ and/or TNFα. NK cells may further be characterized by the expression of activating and inhibitory receptors referred to as killer immunoglobulin-like receptors (KIRs). Other activating receptors that NK cells may express include NKG2D, the CD94/NKG2 receptors including NKG2CNKG2E and NKG2F and natural cytotoxicity receptors (NCRs) including NKp30, NKp44, and NKp46. Other inhibitory receptors include CD94/NKG2 receptors including NKG2A, and NKG2B. The differentiation of NK cells from PSC or HSPC (e.g. PSC-derived HSPC) is usually intermediated by one or more progenitor populations, such as PSC-derived mesodermal precursors and/or lymphoid progenitor cells (e.g. PSC-derived lymphoid progenitors).

Where used herein, the term “T cell” refers to a type of lymphocyte of the hematopoietic lineage that may derive from a HSPC. More specifically, T cells may derive from multilymphoid progenitors (MLPs) or common lymphoid progenitors (CLPs). T cells are typically characterized by: the absence of NK-, B-, and erythromyeloid-specific markers; the expression of CD3, and TCRαβ (or TCRγδ), and CD4 or CD8; and their effector functions. Although, some subsets of T cells may express characteristics of both T and NK cells, and may or may not express TCRαβ or TCRγδ and may or may not also express CD4, CD8, CD56, CD16 and NK1.1. More specifically, effector functions of T cells may include the production of cytokines, killing of infected or tumor target cells, or the stimulation of other hematopoietic cell types. T cells may further be characterized by the expression of CD8a, CD8B, CD45RA and CD27. The differentiation of T cells from PSC or HSPC (e.g. PSC-derived HSPC) is usually intermediated by one or more progenitor populations, such as PSC-derived mesodermal precursor and/or lymphoid progenitor cells (e.g. PSC-derived lymphoid progenitors).

### Methods

The methods of this disclosure encompass those steps for differentiating hematopoietic progenitors, and other downstream hematopoietic cell types, from PSC or early mesoderm cells. The methods of this disclosure may also encompass those steps for differentiating PSC to T cells, NK cells, and B cells via one or more precursor cell populations. The methods disclosed herein for differentiating hematopoietic progenitors, and other downstream hematopoietic cell types, are preferably in vitro and/or ex vivo methods.

A method of differentiating hematopoietic progenitor cells comprises culturing a population of early mesoderm cells (e.g. precursor cells of hematopoietic progenitors) in a first medium comprising a first basal medium supplemented with one or more cytokine(s) or growth factor(s). In one embodiment, the one or more cytokine(s) or growth factor(s) is thrombopoeitin (TPO), stem cell factor (SCF) and FMS-like tyrosine kinase 3 ligand (FLT3L). In one embodiment, the first medium excludes any combination of exogenously added agonists selected from the list consisting of an agonist of BMP signaling, an agonist of fibroblast growth factor (FGF) signaling, and an agonist of vascular endothelial growth factor (VEGF) signaling. In another embodiment, the first medium consists of or consists essentially of a basal medium supplemented with one or more of thrombopoeitin (TPO), stem cell factor (SCF) and FMS-like tyrosine kinase 3 ligand (FLT3L).

In one embodiment, the population of early mesoderm cells are precursors of hematopoietic stem and progenitor cells, and they are isolated from an appropriate tissue, such as embryos (e.g. aorta-gonad-mesonephros). In one embodiment, the mesoderm precursor cells correspond to a specialized subset of vascular endothelium known as hemogenic endothelium. In some embodiments, the population of early mesoderm cells (e.g. the mesoderm precursor cells) are differentiated from PSC. Regardless of whether or not the population of early mesoderm cells are PSC-derived or isolated from early stage embryos, they may express one or more of KDR, CD56 (NCAM), or Brachyury, but may lack expression of markers such as CD326 (EpCAM).

First media of this disclosure include any media which may be used to differentiate hematopoietic progenitor cells (or hemogenic endothelium, HE) from mesoderm cells. HE cells are endothelial cells located in the aorta-gonad-mesonephros (AGM) region in the embryo and are capable of differentiating to hematopoietic progenitors through a process called endothelial-to-hematopoietic (EHT) transition. Differentiating the population of early mesoderm cells to hematopoietic progenitor cells in first media may go through one or more populations of intermediates (e.g. hemogenic endothelium). Or, such first media may differentiate a population of early mesoderm cells directly or indirectly to hematopoietic progenitor cells.

First media of this disclosure may contain serum or may be serum-free. Preferably, first media of this disclosure are serum-free. If the media are serum-free, it may be necessary to include in such media a serum replacement supplement, such as BIT 9500 Serum Substitute (STEMCELL Technologies, Catalogue #09500), or other commercially available serum replacement solutions. Alternatively, components ordinarily present in serum that are needed for culturing or differentiating any cells of this disclosure may be individually added at an acceptable concentration to the media, such as an albumin (whether recombinant or otherwise).

First media of this disclosure will include a first basal medium that is formulated as appropriate to culture/differentiate the cells of this disclosure (e.g. hematopoietic progenitor cells, or HSPC, and potentially downstream lymphoid progenitors, NK cells, T cells, B cells). Thus, first basal medium may be any basal medium that is supportive of culturing/differentiating cells of the hematopoietic lineage. By way of non-limiting example, fist basal medium may be STEMdiff Hematopoietic—EB Basal Medium (STEMCELL Technologies, Catalogue #100-171), STEMdiff Hematopoietic Basal Medium (STEMCELL Technologies, Catalogue #05311), STEMdiff™ APEL™2 Medium (STEMCELL Technologies, Catalogue #05270), or any other commercially available basal medium fit for the purpose. Common components used to formulate such first basal media may include salts, buffers, lipids, amino acids, trace elements, certain proteins, etc. In one embodiment, first basal media is formulated to optimally support the differentiation of hematopoietic progenitor cells (or hemogenic endothelium, HE) from a population of early mesoderm cells.

In one embodiment, first media of this disclosure may include one or more cytokines or growth factors known to influence early stages of hematopoietic differentiation. The supplement(s) added to the first basal media may vary depending on the specific type of cell to be cultured. In general, a non-exhaustive list of potential supplements includes at least one of: one or more cytokines; one or more growth factors; or other proteins.

In one embodiment, the one or more cytokine or growth factor may be IL-2, IL-3, IL-6, IL-7, IL-11, IL-15, SCF, FLT3L, TPO, EPO, and IGF-1 or IGF-2. In one embodiment first media is supplemented with each of IL-2, IL-3, IL-6, IL-7, IL-11, IL-15, SCF, FLT3L, TPO, EPO, and IGF-1 or IGF-2. In one embodiment, first media is supplemented with one or more of IL-2, IL-3, IL-6, IL-7, IL-11, IL-15, SCF, FLT3L, TPO, EPO, and IGF-1 or IGF-2. In embodiments of first media including one or more of IL-2, IL-3, IL-6, IL-7, IL-11, IL-15, SCF, FLT3L, TPO, EPO, and IGF-1 or IGF-2, such cytokines may respectively be present at concentrations between about 1-1000 ng/mL, or about 1-100 ng/mL, or about 5-50 ng/mL. In embodiments where small molecule analogues of one or more of the foregoing cytokines are included in first media, they would typically be used at lower concentrations.

In some embodiments, any one or more of L-2, IL-3, IL-6, IL-7, IL-11, IL-15, SCF, FLT3L, TPO, EPO, and IGF-1 or IGF-2 may not be included, however, the efficiency of the media may be compromised. In one embodiment, the inclusion of IL-15 in first media is dispensable. In one embodiment, first media comprises one or more of SCF, FLT3L and TPO.

In one embodiment, first media may also include one or more other growth factors commonly used in the culture or differentiation of hematopoietic cells, including hematopoietic progenitor cells. For example, first media may include one or more of an agonist of BMP signaling, an agonist of FGF signaling, and an agonist of VEGF signaling. In one embodiment, first media does not include one, some or all of an agonist of BMP signaling, an agonist of FGF signaling, and an agonist of VEGF signaling. Said a different way, first media may exclude any combination of exogenously added agonists selected from the list consisting of an agonist of BMP signaling, an agonist of fibroblast growth factor (FGF) signaling, and an agonist of vascular endothelial growth factor (VEGF) signaling.

In one embodiment, first media either includes or does not include an exogenously added agonist of BMP signaling. If included, the agonist of BMP signaling may be any known agonist of BMP signaling. The agonist of BMP signaling may be endogenously expressed in and isolated from a producing cell, or the agonist of BMP signaling may be recombinant and expressed and isolated from a producing cell. Or, the agonist of BMP signaling may be a small molecule. In one embodiment, the agonist of BMP signaling is a bone morphogenic protein of any species, such as mouse or human BMP4.

A concentration of the agonist of BMP signaling in first media may range between about 0.05 ng/mL and 5 μg/mL, between about 0.1 ng/mL and 1 μg/mL, between about 0.5 ng/mL and 500 ng/mL, between about 1 ng/mL and 100 ng/mL, or between about 5 and 50 ng/mL.

In one embodiment, first media either includes or does not include an exogenously added agonist of FGF signaling. If included, the agonist of FGF signaling may be any known fibroblast growth factor. The fibroblast growth factor may be endogenously expressed in and isolated from a producing cell, or the fibroblast growth factor may be recombinant and expressed and isolated from a producing cell. In one embodiment, the fibroblast growth factor is human or mouse FGF2 (also known as basic FGF, bFGF).

A concentration of the fibroblast growth factor in first media may range between about 0.05 ng/mL and 5 μg/mL, between about 0.1 ng/mL and 1 μg/mL, between about 0.5 ng/mL and 500 ng/mL, between about 1 ng/mL and 100 ng/mL, or between about 5 and 50 ng/mL.

In one embodiment, first media either includes or does not include an exogenously added agonist of VEGF signaling. If included, the agonist of VEGF signaling may be any known vascular endothelial growth factor. The vascular endothelial growth factor may be endogenously expressed in and isolated from a producing cell, or the fibroblast growth factor may be recombinant and expressed and isolated from a producing cell. In one embodiment, the vascular endothelial growth factor is human or mouse VEGF.

A concentration of the vascular endothelial growth factor in first media may range between about 0.05 ng/mL and 5 μg/mL, between about 0.1 ng/mL and 1 μg/mL, between about 0.5 ng/mL and 500 ng/mL, between about 1 ng/mL and 100 ng/mL, or between about 5 and 50 ng/mL.

In one embodiment, first media excludes one or more exogenously added BMP4, FGF2, and VEGF.

In some embodiments, first media does not include an Activin (e.g. Activin A), a Wnt signaling agonist, such as CHIR99021, and/or an inhibitor of Activin receptor-like kinase, such as SB431542.

Further, first media of this disclosure may synergize with additional supplements for supporting the culture of cells. For example, on the one hand, stromal or feeder cells may be used together with cell culture media of this disclosure. Non-exhaustive examples of such cells include the embryonic liver cell line EL08.1D2, AFTO24 cells, OP9 cells, MS-5 or M2-10B4 cells, mouse embryonic fibroblasts or stromal cells from embryonic aorta-gonad mesonephros (AGM). On the other hand, stroma- and/or feeder-free culture approaches may be used together with cell culture media of this disclosure. In some embodiments of such approaches, medium conditioned by the culture of stromal/feeder cells may be used, or such a system may utilize a stroma/feeder cell replacement. A stroma/feeder cell replacement may comprise one or more defined components that provide appropriate signals or attachment sites to cells in culture. Such components may be included in first media or in a coating applied to an inner culture surface of a culture vessel or on solid surfaces suspended in a cell culture media, such as on particles, beads, microcarriers, or the like. Non-exhaustive examples of such components may include fibronectin coatings, gelatin coatings, collagen coatings, or coatings such as StemSpan™ Lymphoid Differentiation Coating Supplement (STEMCELL Technologies, Catalogue #09925) or Matrigel (Corning). Or, stroma/feeder cell replacement may provide such components in soluble form within a cell culture media, such as by supplementation or as a medium previously conditioned by stromal/feeder cells.

In one embodiment, a method of differentiating hematopoietic progenitor cells from a population of early mesoderm cells may further comprise culturing the early mesoderm cells (e.g. mesoderm precursor cells) under stroma-free conditions and/or feeder-free conditions. In one embodiment, a method of differentiating hematopoietic progenitor cells from a population of early mesoderm cells may further comprise culturing the mesoderm precursor cells under conditions devoid of exogenously added stroma- and/or feeder-cells.

Culturing (in an appropriate medium, such as first media, as described above) the population of early mesoderm cells, such as the PSC-derived mesoderm precursor cells, may be for any period of time that does not impact their viability or capacity to differentiate to downstream lineages. In one embodiment, a method of differentiating hematopoietic progenitor cells from a population of early mesoderm cells further comprises culturing the cells in a first medium for between about 2 days and 21 days, or between about 4 days and 14 days. In one embodiment, a method of differentiating hematopoietic progenitor cells from a population of early mesoderm cells further comprises culturing the cells in a first medium for between about 6 days and 10 days.

It was surprisingly shown by the inventors that an absence of one, some, or all of an exogenously added BMP signaling agonist, FGF signaling agonist and VEGF signaling agonist in first media (as otherwise described herein) for differentiating hematopoietic progenitor cells from a population of early mesoderm cells did not markedly decrease efficiency of the differentiation. In some cases, excluding any of the foregoing agonists (or any combination) may have a beneficial impact on the efficiency of differentiating hematopoietic progenitor cells from a population of early mesoderm cells. For example, absence of an exogenously added VEGF signaling agonist may increase the frequency and/or yield of differentiated hematopoietic progenitor cells in comparison to first media including an exogenous VEGF signaling agonist. Furthermore, hematopoietic progenitor cells differentiated in a first media not including an exogenously added VEGF signaling agonist may be less adherent, thereby producing increased frequency and yield of downstream lymphoid progenitors (and further arising T cells, B cells, and/or NK cells), in comparison to a first media including an exogenously added VEGF signaling agonist. Similarly, absence of an exogenously added FGF or BMP signaling agonist may increase the frequency and/or yield of differentiated hematopoietic progenitor cells in comparison to first media including an exogenously added FGF or BMP signaling agonist. Whereas excluding from first media either a FGF or BMP signaling agonist may result in increased in hematopoietic progenitor cells, it may be that such cells have an impaired capacity to differentiate to downstream lymphoid progenitors. Surprisingly, though, hematopoietic progenitor cells differentiated in a first media excluding each of an agonist of VEGF signaling, an agonist of FGF signaling, and an agonist of BMP signaling may have an increased (or equivalent) capacity to further differentiate to lymphoid progenitors (in comparison to conditions including each of the three). Other benefits of identifying minimal media conditions for differentiating hematopoietic progenitor cells (and downstream lymphoid progenitors) are that manufacturing costs may be significantly reduced, that complexity is reduced, and that reduced complexity may provide fewer regulatory hurdles.

Accordingly, this disclosure also encompasses media for differentiating hematopoietic progenitor cells. In one embodiment, this disclosure encompasses media for differentiating PSC-derived hematopoietic progenitor cells. In one embodiment, the PSC are mammalian, and in one embodiment the PSC are human. A media for differentiating hematopoietic progenitor cells may be referred to as a first media, and such first media may comprise any of the formulation details described above. In one embodiment, first media is serum-free and is used in feeder/stroma-free workflows. In one embodiment, first media comprises a basal medium supplemented with one or more of thrombopoeitin (TPO), stem cell factor (SCF) and FMS-like tyrosine kinase 3 ligand (FLT3L), and excluding any combination of exogenously added agonists selected from the list consisting of an agonist of BMP signaling, an agonist of fibroblast growth factor (FGF) signaling, and an agonist of vascular endothelial growth factor (VEGF) signaling.

An absence of one, some, or all of the BMP, FGF and VEGF agonists from first media may mean that cells cultured in first media (and in contact with stroma cells or a coating material in stroma-free workflows) are nevertheless exposed to low levels or levels below detection of such agonists, if for example the stroma cells or stroma replacement, or the cells themselves, introduce the growth factor. However, neither the PSC nor the stroma cells (if present) are engineered to ectopically express or overexpress such agonists. Accordingly, to the knowledge of the inventors, this is the first disclosure reporting efficient differentiation of hematopoietic progenitor cells from PSC-derived mesoderm precursor cells, under serum- and stroma-free conditions, in the absence of one, some, or all of BMP, FGF, and VEGF signaling agonists.

In one embodiment, a method of differentiating PSC-derived hematopoietic progenitor cells from a population of early mesoderm cells may further comprise culturing the PSC in a derivation medium comprising a second basal medium and one or more of an agonist of BMP signaling (as described above), an agonist of FGF signaling (as described above), and an agonist of vascular endothelial growth factor (VEGF) signaling, to obtain a differentiated population of PSC-derived early mesoderm cells.

Derivation media of this disclosure include any media that may be used to differentiate a population of early mesoderm cells from PSC. Deriving a population of early mesoderm cells from PSC in derivation medium may go through one or more populations of intermediates. Or, derivation media may differentiate a population of PSC directly or indirectly to a population of early mesoderm cells.

Derivation media of this disclosure may contain serum or may be serum-free. Preferably, derivation media of this disclosure are serum-free. If the media are serum-free, it may be necessary to include in such media a serum replacement supplement, such as BIT 9500 Serum Substitute (STEMCELL Technologies, Catalogue #09500), or other commercially available serum replacement solutions. Alternatively, components ordinarily present in serum that are needed for culturing or differentiating any cells of this disclosure may be individually added at an acceptable concentration to the media, such as an albumin (whether recombinant or otherwise).

Derivation media of this disclosure will include a second basal medium that is formulated as appropriate to differentiate the cells of this disclosure (e.g. PSC, and potentially downstream hematopoietic progenitor cells, lymphoid progenitors, NK cells, T cells, B cells). Second basal medium may be any basal medium which is supportive of culturing PSC and/or cells of the hematopoietic lineage, including progenitors thereof (e.g. early mesoderm cells). By way of non-limiting example, second basal medium may be STEMdiff Hematopoietic—EB Basal Medium (STEMCELL Technologies, Catalogue #100-171), STEMdiff Hematopoietic Basal Medium (STEMCELL Technologies, Catalogue #05311), STEMdiff™ APEL™2 Medium (STEMCELL Technologies, Catalogue #05270 or any other commercially available basal medium fit for the purpose. Common components used to formulate such first basal media may include salts, buffers, lipids, amino acids, trace elements, certain proteins, etc.

In one embodiment, second basal medium is the same, or essentially the same, as first basal medium. In one embodiment, second basal medium is different from first basal medium.

In one embodiment, derivation media may comprise one or more growth factors known to influence early stages of hematopoietic differentiation. For example, derivation media may include one or more of an agonist of BMP signaling, an agonist of FGF signaling, and an agonist of VEGF signaling.

In one embodiment of derivation media, the agonist of BMP signaling may be any known agonist of BMP signaling. The agonist of BMP signaling may be endogenously expressed in and isolated from a producing cell, or the agonist of BMP signaling may be recombinant and expressed and isolated from a producing cell. Or, the agonist of BMP signaling may be a small molecule. In one embodiment, the agonist of BMP signaling is a bone morphogenic protein. In one embodiment, the agonist of BMP signaling is BMP4. In one embodiment the agonist of BMP signaling is human or mouse BMP4.

If included, a concentration of the agonist of BMP signaling in derivation media may range between about 0.05 ng/mL and 5 μg/mL, between about 0.1 ng/mL and 1 μg/mL, between about 0.5 ng/mL and 500 ng/mL, between about 1 ng/mL and 100 ng/mL, or between about 5 and 50 ng/mL.

In one embodiment of derivation media, the agonist of FGF signaling may be any known fibroblast growth factor. The fibroblast growth factor may be endogenously expressed in and isolated from a producing cell, or the fibroblast growth factor may be recombinant and expressed and isolated from a producing cell. In one embodiment, the fibroblast growth factor is human or mouse FGF2 (also known as basic FGF, bFGF). In some embodiments, the fibroblast growth factor consists of a single isoform variant. In some embodiments, the fibroblast growth factor comprises more than one isoform variant.

If included, a concentration of the fibroblast growth factor agonist in derivation media may range between about 0.05 ng/mL and 5 μg/mL, between about 0.1 ng/mL and 1 μg/mL, between about 0.5 ng/mL and 500 ng/mL, between about 1 ng/mL and 100 ng/mL, or between about 5 and 50 ng/mL.

In one embodiment of derivation media, the agonist of VEGF signaling may be any known vascular endothelial growth factor, or any splicing isoform thereof. The vascular endothelial growth factor or splice variant(s) may be endogenously expressed in and isolated from a producing cell, or the vascular endothelial growth factor or splice variant(s) may be recombinant and expressed and isolated from a producing cell. In one embodiment, a single isoform of vascular endothelial growth factor is included in derivation media. In one embodiment, multiple isoforms of vascular endothelial growth are included in derivation media. Human vascular endothelial growth factor is known to be spliced in multiple ways. For example, alternatively spliced isoforms of vascular endothelial growth factor include isoforms of 121, 145, 165, 183, 189, and 206 amino acids in length. The most potent and abundant isoforms may include the 121, 165 and 189 amino acid-long variants.

If included, a concentration of the vascular endothelial growth factor agonist in derivation media may range between about 0.05 ng/mL and 5 μg/mL, between about 0.1 ng/mL and 1 μg/mL, between about 0.5 ng/mL and 500 ng/mL, between about 1 ng/mL and 100 ng/mL, or between about 5 and 50 ng/mL.

Derivation media may further include other cytokines or growth factors known to influence early stages of hematopoietic differentiation, such as an Activin (e.g. Activin A), a Wnt signaling agonist (e.g. CHIR99021), and/or a TGFβ pathway inhibitor (e.g. SB431542), and/or an Activin.

Further, derivation media of this disclosure may synergize with additional supplements for the culture of the cells. For example, on the one hand, stromal and/or feeder cells may be used together with cell culture media of this disclosure. Non-exhaustive examples of such cells include the embryonic liver cell line EL08.1D2, AFTO24 cells, OP9 cells, MS-5 or M2-10B4 cells, mouse embryonic fibroblasts or stroma cells from embryonic AGM. On the other hand, stroma- and/or feeder-free culture approaches may be used together with cell culture media of this disclosure. Stroma- and/or feeder-free culture systems may utilize medium previously conditioned by stromal cells, or such a system may utilize a stroma cell replacement. Such systems may utilize medium previously conditioned by stromal/feeder cells, or such a system may utilize a stroma/feeder cell replacement. A stroma/feeder cell replacement may comprise one or more defined components that provide appropriate signals or attachment sites to cells in culture. Such components may be included in derivation media or in a coating applied to an inner culture surface of a culture vessel or on solid surfaces suspended in a cell culture media, such as on particles, beads, microcarriers, or the like. Non-exhaustive examples of such components may include fibronectin coatings, gelatin coatings, collagen coatings, an immobilized Notch ligand, or coatings such as StemSpan™ Lymphoid Differentiation Coating Supplement (STEMCELL Technologies, Catalogue #09925) or Matrigel (Corning). Or, stroma/feeder cell replacement may provide such components in soluble form within a cell culture media, such as by supplementation or as a medium previously conditioned by stromal/feeder cells.

In one embodiment, a method of differentiating a population of early mesoderm cells from PSC may further comprise culturing the PSC under stroma-free conditions and/or feeder-free conditions. In one embodiment, a method of differentiating a population of early mesoderm cells from PSC may further comprise culturing the PSC under conditions devoid of exogenously added stroma- and/or feeder-cells.

In one embodiment, a method of differentiating a population of early mesoderm cells from PSC may further comprise dissociating PSCs into clumps, or single cells, and plating such cells in the derivation medium.

In one embodiment, a method of differentiating a population of early mesoderm cells from PSC may further comprise forming the PSC into aggregates prior to culturing the PSC in the derivation medium.

In one embodiment, a method of differentiating a population of early mesoderm cells from PSC may further comprise forming the PSC into aggregates in the derivation medium.

PSC may be formed into aggregates using any known approach. For example, aggregates of PSC may be formed by depositing a desired number of PSC into the bottom a tube or a well of a cell culture plate. Or, aggregates may be formed by depositing a desired number of PSC into a well of an Aggrewell™ microwell device, to ensure the efficient and reproducible formation of uniformly sized aggregates of PSC.

In one embodiment, the number of PSC used to form the aggregates is between about 1 and 100,000. In one embodiment, the number of PSC used to form the aggregates is between about 10 and 10,000. In one embodiment, the number of PSC used to form the aggregates is between about 100 and 1,000.

Therefore, in one embodiment, the aggregates of PSC may be formed in a microwell device. In one embodiment, the aggregates of PSC are formed from about 1000 cells or about 500 cells.

Culturing (in an appropriate medium, such as derivation media, as described above) the PSC to generate mesoderm precursors, may be for any period of time that does not impact their viability or capacity to differentiate to downstream lineages. In one embodiment, a method of differentiating hematopoietic progenitor cells from a population of early mesoderm cells further comprises culturing the PSC to obtain a differentiated population of PSC-derived mesoderm cells in a derivation medium for between about 1 days and 7 days. In one embodiment, a method of differentiating a population of early mesoderm cells from PSC further comprises culturing the PSC in a derivation medium for between about 2 days and 5 days.

In one embodiment, PSC may be differentiated into a population of early mesoderm cells in a derivation medium comprising a second basal medium, and one or more of an agonist of BMP signaling, an agonist of FGF signaling, and an agonist of vascular endothelial growth factor (VEGF) signaling to obtain a differentiated population of PSC-derived mesoderm cells. In one embodiment, derivation media include each of a second basal medium and agonists of BMP, FGF, and VEGF signaling.

Accordingly, based on the foregoing, this disclosure also encompasses derivation media, as described hereinabove. Further, derivation media and first media may be included in a kit or a system for differentiating PSC to hematopoietic progenitors through a population of early mesoderm cells.

In one embodiment, a method of differentiating hematopoietic progenitor cells from PSC-derived mesoderm precursors, further comprises obtaining lymphoid progenitor cells after dissociating the hematopoietic progenitors and culturing the dissociated hematopoietic progenitor cells in a lymphoid differentiation medium under serum-free and/or stroma-free conditions.

Where aggregates of PSC are used to differentiate a population of early mesoderm precursors and such PSC-derived mesoderm precursors are used to differentiate hematopoietic progenitor cells, the hematopoietic progenitor cells may be dissociated using any known or developed means. Various dissociation reagents are commercially available, such as Accutase™ (STEMCELL Technologies), TrypLE™ Express (ThermoFisher Scientific), Gentle Cell Dissociation Reagent, GCDR (STEMCELL Technologies), or trypsin. It may also be desirable to combine different dissociation reagents. For example, as shown herein, Collagenase II & TrypLE™ or Collagenase IV & TrypLE™ may be combined to dissociate the hematopoietic progenitor cells.

In one embodiment, following dissociating the hematopoietic progenitor cells, it may be desirable to enrich for hematopoietic progenitor cells within the sample. Various commercial reagents are known, including EasySep™ Human CD34 Positive Selection Kit II (STEMCELL Technologies). Or, the hematopoietic progenitors (or hemogenic endothelial cells) may be enriched by FACS sorting.

A lymphoid differentiation medium of this disclosure includes any media that may be used to differentiate hematopoietic progenitor cells or hemogenic endothelial cells to lymphoid progenitor cells. Such lymphoid differentiation medium may refer to differentiation of PSC-derived hematopoietic progenitor cells to lymphoid progenitor cells, which may include the derivation of one or more populations of intermediates therebetween. Or, such lymphoid differentiation medium may differentiate a population of PSC-derived hematopoietic progenitor cells directly or indirectly to one or more subsets of lymphoid progenitor cells.

A lymphoid differentiation medium of this disclosure may contain serum or may be serum-free. Preferably, a lymphoid differentiation medium of this disclosure are serum-free. If the medium is serum-free, it may be necessary to include in such medium a serum replacement supplement, such as BIT 9500 Serum Substitute (STEMCELL Technologies, Catalogue #09500), or other commercially available serum replacement solutions. Alternatively, components ordinarily present in serum that are needed for culturing or differentiating any cells of this disclosure may be individually added at an acceptable concentration to the media.

A lymphoid differentiation medium of this disclosure will include a third basal medium that is formulated as appropriate to differentiate the cells of this disclosure (e.g. hematopoietic progenitor cells to lymphoid progenitors). Third basal medium may be any basal medium that is supportive of culturing cells of the hematopoietic lineage. By way of non-limiting example, third basal medium may be StemSpan™ SFEM (STEMCELL Technologies, Catalogue #09650), StemSpan™ SFEM II (STEMCELL Technologies, Catalogue #09655), StemSpan™-ACF (STEMCELL Technologies, Catalogue #09855), StemSpan™ H3000 (STEMCELL Technologies, Catalogue #09850), or any other commercially available basal medium fit for the purpose. Common components used to formulate such third basal media may include salts, buffers, lipids, amino acids, trace elements, certain proteins, etc.

In one embodiment, third basal medium is the same as either second basal medium or the first basal medium, or both, or essentially the same, as either second basal medium or the first basal medium, or both. In one embodiment, third basal medium is different from either the second basal medium or the first basal medium, or both.

In one embodiment, the lymphoid differentiation medium may need to be further supplemented in order to support differentiation of the hematopoietic progenitor cells. The supplement(s) added to the basal media may vary depending on the specific type of cell to be cultured. In general, a non-exhaustive list of potential supplements includes one or more cytokines, one or more growth factors, or other proteins.

Specifically, IL-2, IL-3, IL-6, IL-7, IL-11, IL-15, SCF, FLT3L, TPO, EPO, and IGF-1 or IGF-2 may be included in lymphoid differentiation media of this disclosure. In one embodiment lymphoid differentiation media is supplemented with each of IL-2, IL-3, IL-6, IL-7, IL-11, IL-15, SCF, FLT3L, TPO, EPO, and IGF-1 or IGF-2. In one embodiment, lymphoid differentiation media is supplemented with one or more of IL-2, IL-3, IL-6, IL-7, IL-11, IL-15, SCF, FLT3L, TPO, EPO, and IGF-1 or IGF-2. In embodiments of lymphoid differentiation media including one or more of IL-2, IL-3, IL-6, IL-7, IL-11, IL-15, SCF, FLT3L, TPO, EPO, and IGF-1 or IGF-2, such cytokines may respectively be present at concentrations between about 1-1000 ng/mL, or about 1-100 ng/mL, or about 5-50 ng/mL. If a small molecule is also included, they typically are used at lower concentrations. For example, small molecules such as the Wnt signaling agonist CHIR99021 or the TGFβ pathway inhibitor SB431542 may be used at concentrations between 0.1 nM-30 uM.

In some embodiments, any one or more of IL-2, IL-3, IL-6, IL-7, IL-11, IL-15, SCF, FLT3L, TPO, EPO, and IGF-1 or IGF-2 may not be included in the lymphoid differentiation medium, however, the efficiency of the lymphoid differentiation medium may be compromised.

In one embodiment, a method of differentiating hematopoietic progenitor cells from mesoderm cells (with subsequent differentiation of the hematopoietic progenitor cells to lymphoid progenitor cells), may further comprise obtaining T cells after culturing the lymphoid progenitor cells in a T cell maturation medium.

In one embodiment T cell maturation medium may include any one or more of IL-2, IL-3, IL-6, IL-7, IL-11, IL-15, SCF, FLT3L, TPO, EPO, and IGF-1 or IGF-2. In one embodiment, one, some or all of IL-2, IL-3, IL-6, IL-7, IL-11, IL-15, SCF, FLT3L, TPO, EPO, and IGF-1 or IGF-2 may not be included T cell maturation medium; however, the efficiency of such media may be compromised. For example, IL-15 may be excluded from T cell maturation medium without significantly impacting the efficiency of such medium.

In one embodiment, the T cells are CD4⁺CD8⁺ double positive T cells and may express CD3 and TCRαβ. In one embodiment, the CD4⁺CD8⁺ double positive T cells may be further matured to CD8⁺ single positive T cells.

In one embodiment, a method of differentiating hematopoietic progenitor cells from mesoderm cells (with subsequent differentiation of the hematopoietic progenitor cells to lymphoid progenitor cells), may further comprise obtaining NK cells after culturing the lymphoid progenitor cells in an NK cell generation medium.

In one embodiment, NK cell generation medium may include any one or more of IL-2, IL-3, IL-6, IL-7, IL-11, IL-15, SCF, FLT3L, TPO, EPO, and IGF-1 or IGF-2. In one embodiment, one, some or all of IL-2, IL-3, IL-6, IL-7, IL-11, IL-15, SCF, FLT3L, TPO, EPO, and IGF-1 or IGF-2 may not be included NK cell generation medium; however, the efficiency of such media may be compromised. For example, IL-15 is a critical component of NK cell generation medium in some embodiments.

In one embodiment, the NK cells are CD56+NK cells. In one embodiment, the NK cells are functional. In one embodiment, the NK cells produce IFN-γ. In the same or different embodiment, the NK cells are cytotoxic. In the same or different embodiment, the NK cells express CD107, and more specifically CD107a.

In addition to disclosing methods of differentiating PSC to a population of early mesoderm cells (e.g. precursor cells), PSC-derived mesoderm precursors to hematopoietic progenitor cells, PSC-derived hematopoietic progenitors to lymphoid progenitors, and PSC-derived lymphoid progenitors to various lymphoid lineages, the various media disclosed herein may be encompassed in a stand-alone format or included in a system for the step-wise differentiation of PSC to lymphoid progenitors and beyond, whether or not under serum- and/or stroma-free conditions.

In some embodiments, the entire system is performed under serum- and/or stroma-free conditions. In some embodiments, only certain aspects of the system are performed under serum- and/or stroma-free conditions. For example, but without limiting the generality of the foregoing, differentiating PSC to a population of early mesoderm cells, PSC-derived mesoderm precursors to hematopoietic progenitor cells, and PSC-derived hematopoietic progenitors to lymphoid progenitors are performed under serum- and/or stroma-free conditions, while further downstream stages may or may not. Conversely, earlier stages of the system may or may not be performed under serum- and/or stroma-free conditions, while subsequent stages are performed under serum- and/or stroma-free conditions.

In one embodiment, such system(s) or kit(s) may include one, two, three, four, or five of the following components: a first culture system for differentiating PSC to a population of early mesoderm cells; a second culture system for differentiating PSC-derived mesoderm cells to hematopoietic progenitors cells; a third culture system for differentiating PSC-derived hematopoietic progenitors cells to one or more subsets of lymphoid progenitor cells; a fourth culture system for differentiating PSC-derived lymphoid progenitor cells to B cells; a fifth culture system for differentiating PSC-derived lymphoid progenitor cells to T cells; a sixth culture system for differentiating PSC-derived lymphoid progenitor cells to NK cells; a coating substrate; a first kit for enriching, either positively or negatively, a population of early mesoderm cells (e.g. precursor cells); a second kit for enriching, either positively or negatively, a population of hematopoietic progenitor cells; a third kit for enriching, either positively or negatively, a population of lymphoid progenitor cells; a fourth kit for enriching, either positively or negatively, a population of B cells; a fifth kit for enriching, either positively or negatively, a population of T cells; and a sixth kit for enriching, either positively or negatively, a population of NK cells

The following non-limiting examples are illustrative of the present disclosure.

### EXAMPLES

### Example 1: Maintenance of PSC

Human PSCs (hPSCs) were maintained on Matrigel™ coated plates in either mTeSR™1 (STEMCELL Technologies), TeSR™-E8 (STEMCELL Technologies), or mTeSR™ Plus (STEMCELL Technologies) media for 6-8 days, in accordance with the manufacturer's recommendations. Complete media changes were performed daily. PSC colonies were clump passaged onto freshly coated Matrigel™ plates in maintenance culture of the PSC line. Where the PSC were used for downstream differentiation assays, the colonies were dissociated using ACCUTASE™ (STEMCELL Technologies) to obtain a single cell suspension, in accordance with manufacturer's recommended protocol.

### Example 2: Formation of Aggregates

Prior to obtaining the single cell suspension in accordance with Example 1, 24-well or 6-well Aggrewell™ 400 plates (STEMCELL Technologies) were prepared in accordance with the manufacturer's recommendations, including coating the microwell devices with anti-adherence rinsing solution (STEMCELL Technologies) to reduce adherence of cells to the microwell device (STEMCELL Technologies). Following the recommended incubation, the anti-adherence rinse solution was discarded and each well was rinsed once with an equal volume of DMEM-F12 with 15 mM HEPES.

After preparing the microwell device, the hPSCs dissociated in accordance with Example 1 were seeded into one or more wells of the microwell device in derivation medium (e.g. EB Formation Medium) (STEMdiff™ Hematopoietic—EB Basal Medium, supplemented with STEMdiff™ Hematopoietic—EB Supplement A, STEMCELL Technologies), and 10 μM Y-27632 (STEMCELL Technologies). In embodiments where the 6-well format of the microwell device is used, 2.5 mL of EB Formation Medium was added to a well of the microwell device. Next, a further 2.5 mL of a cell suspension (˜1.4×10⁶ cells/mL) in EB Formation Medium was added to the well containing 2.5 mL of EB Formation Medium and the microwell device was briefly centrifuged and incubated at 37° C. If a 24-well format of the microwell device is used, then the volume per well should be scaled down accordingly to 2 mL/well. The final cell concentration in each well of the microwell device should be ˜3×10⁵ cells/ml, or 6×10¹ cells/well of a 24-well plate or 7×10⁵ cells/mL, or 3.5×10⁶ cells/well of a 6-well plate.

### Example 3: Flow Cytometry and Determination of Cell Count and Yield

At any stage of the differentiation protocols outlined in this disclosure, the samples can be harvested and the phenotypes thereof can be assessed by flow cytometry. The following general protocol equally applies to measuring CD34, CD5, CD7, NK lineage markers, such as CD56, NKp46, NKp44, NKp30, NKG2D, CD16 or KIR, and T lineage markers, such as CD4, CD8, TCRαβ, or CD3.

Briefly, the cell sample was harvested by centrifugation and appropriately washed. The cell sample was then stained with fluorophore-conjugated antibodies against the antigen of choice. Prepared cell samples were analyzed on the CytoFLEX S™ flow cytometer (Beckman-Coulter). Dead cells were excluded by light scatter profile and DRAQ7 staining.

Total viable cell counts were obtained using the NucleoCounter NC250 (Chemometec) in accordance with the manufacturer's recommendations. Cells were diluted, as required, prior to staining with a mixture of acridine orange and DAPI (AO/DAPI). In this mixture, AO labels the cell membrane and DAPI labels nucleic acid in dead/dying cells—together enabling photographic discrimination of viable vs. non-viable cells in the sample. The NC250 software then analyzes the resulting images and reports the cell counts, including viable cell concentration. To calculate the yield of particular cells per input cell, total viable counts were multiplied by the % frequency of the given cell type. For example, to calculate the yield of NK cells per input CD34⁺ cell, the viable cell count is first multiplied by the % CD56⁺ obtained by flow cytometry. This number is then divided by the number of input cells (in the case input CD34⁺ cells) to obtain the final value. Input CD34⁺ cell numbers were obtained by multiplying total cells cultured in one well by frequency of CD34⁺ cells after cell separation. To calculate the yield of CD34⁺ cells per well of AggreWell™, the yield of CD34⁺ cells (before cell separation) per input hPSC was multiplied by the number of hPSC seeded into one well of AggreWell™ (3.5×10⁶/well of 6wp AggreWell™ 400).

### Example 4: Differentiating Aggregates to Hematopoietic Progenitors

On day 2 after forming the aggregates in accordance with Example 2, 2.5 mL of medium in each well of the microwell device was carefully removed and discarded without disturbing the aggregates. A 2.5 mL volume of fresh derivation medium (e.g. EB Medium A) (STEMdiff™ Hematopoietic—EB Basal Medium (STEMCELL Technologies) supplemented with STEMdiff™ Hematopoietic—EB Supplement A (STEMCELL Technologies)) was added to each well and the microwell device was returned to incubate at 37° C.

On day 3 when mesoderm intermediates (e.g. population of early mesoderm cells) are formed, 2.5 mL of medium in each well of the microwell device was carefully removed and discarded without disturbing the aggregates. A 2.5 mL volume of fresh first medium (e.g. EB Medium B) (STEMdiff™ Hematopoietic—EB Basal Medium (STEMCELL Technologies) supplemented with STEMdiff™ Hematopoietic—EB Supplement B (STEMCELL Technologies)) was added to each well and incubated at 37° C. to differentiate the mesodermal cells to hematopoietic progenitors.

On day 5, the aggregates were harvested from each well of the microwell device and passed through a 37 μm reversible filter (STEMCELL Technologies) to isolate the aggregates on a surface thereof. The filtrate of aggregates was deposited into a fresh tube by inverting the filter over the fresh tube and directing 2.5 mL/well fresh first medium (e.g. EB Medium B) against the mesh (1 mL/well if a 24-well format of the microwell device was used). Thusly obtained aggregates were gently resuspended before adding the full volume to a non-tissue culture-treated plate and then incubated at 37° C. Each well of the 6-well plate was topped up with 2.5 mL fresh EB Medium B (or 1 mL/well of a 24-well plate) on day 7 and then incubated at 37° C. On day 10, a half-medium change with fresh EB Medium B was performed taking care not to disrupt the aggregates and then incubated at 37° C. for a further 2 days.

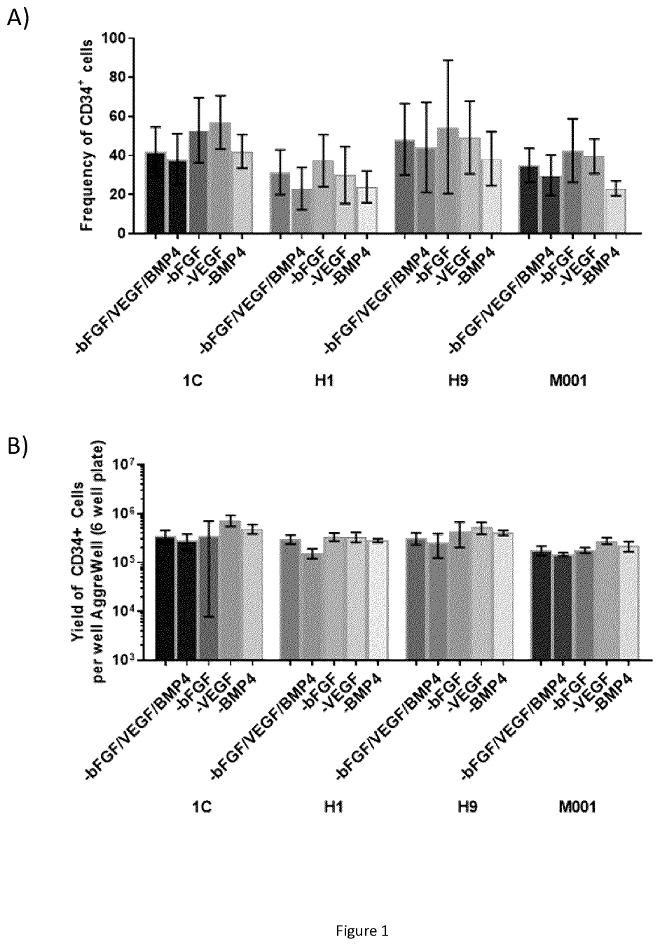

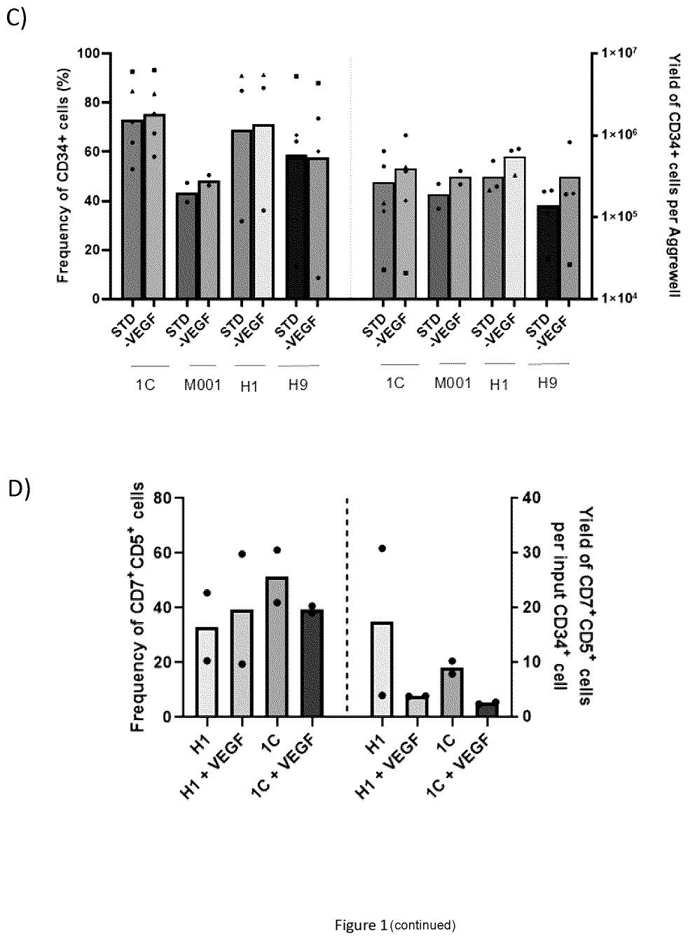

It is commonly believed that agonists of one or more of BMP, FGF, and VEGF signaling are required when differentiating PSC through early mesoderm intermediates to hematopoietic progenitors (Kennedy et al, 2012; Ng et al, 2016), thus it was surprising that excluding these agonists, either alone or in combination, from the differentiation protocol (e.g. first medium) did not negatively impact the output hematopoietic progenitors. In fact, omitting BMP, FGF, and/or VEGF from this stage of the differentiation could increase the output hematopoietic progenitors (FIG. 1A, FIG. 1B, FIG. 1C), while benefiting downstream differentiation of lymphoid progenitors (FIG. 1D), and beyond, such as by reducing the number of contaminating adherent cells in the cultures (data not shown).

### Example 5: Enriching Hematopoietic Progenitors

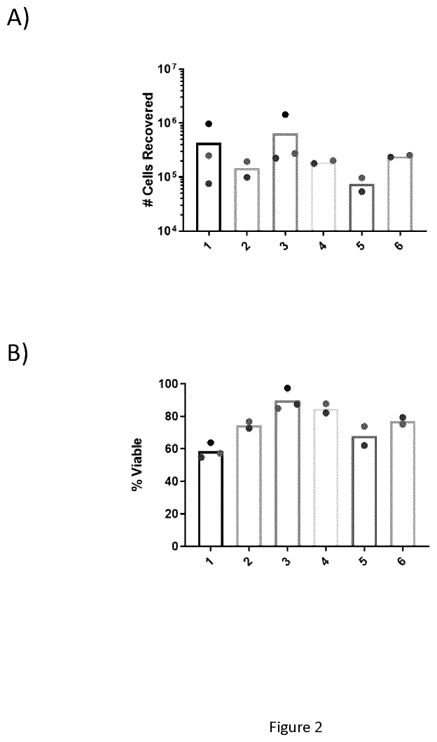

The aggregates of Example 4 were harvested from each well and transferred to individual 15 mL tubes. The tubes were centrifuged at 300 g for 5-10 minutes. The supernatant was aspirated and 1 mL of Collagenase Type II—2500 U/mL (STEMCELL Technologies) (Cat #07418) was added to each tube and incubated at 37° C. for 20 minutes. Following, 3 mL of TryPLE™ Express was added and each tube was incubated for an additional 20 minutes. These dissociation conditions resulted in an increased recovery of CD34⁺ cells and increased yield of viable CD34⁺ cells, as compared to other conventional approaches (FIG. 2)

After the incubation, each tube was topped-up with 6 mL of DMEM/F12 and filtered through a 37 μm filter. The eluate was centrifuged at 300 g× for 5-10 minutes and the supernatant was discarded. The pelleted cells were subjected to a CD34⁺ enrichment protocol using the EasySep™ Human CD34 Positive Selection Kit II (STEMCELL Technologies). The manufacturer's recommendations for the CD34⁺ enrichment were followed except the number of magnetic separations was reduced from four to two.

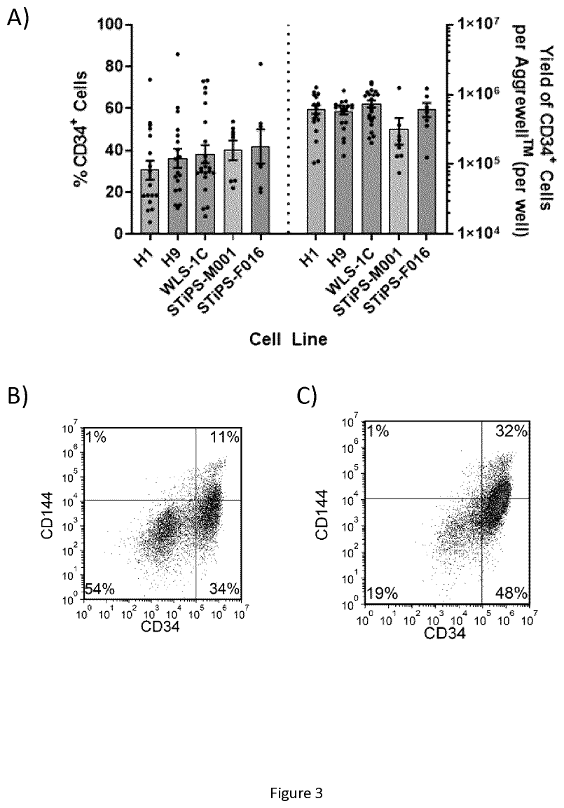

H1, H9, WLS-1C, STiPS-M001, and STiPS-F016 PSC lines efficiently differentiated to CD34⁺ hematopoietic progenitor cells. The mean frequency of CD34⁺ hematopoietic progenitor cells, as calculated in accordance with Example 3, across all PSC lines ranged between 31% and 42% (FIG. 3A). The mean yield per well of a 6-well Aggrewell™ plate of CD34⁺ hematopoietic progenitor cells, as calculated in accordance with Example 3, across all PSC lines ranged between, 3.3×10⁵ and 7.4×10⁵ cells (FIG. 3A). Representative flow cytometry plots of thusly derived CD34⁺ hematopoietic progenitor cells is shown pre-enrichment (FIG. 3B) and post-enrichment (FIG. 3C).

### Example 6: Determining the Multipotency of PSC-Derived Hematopoietic Progenitors

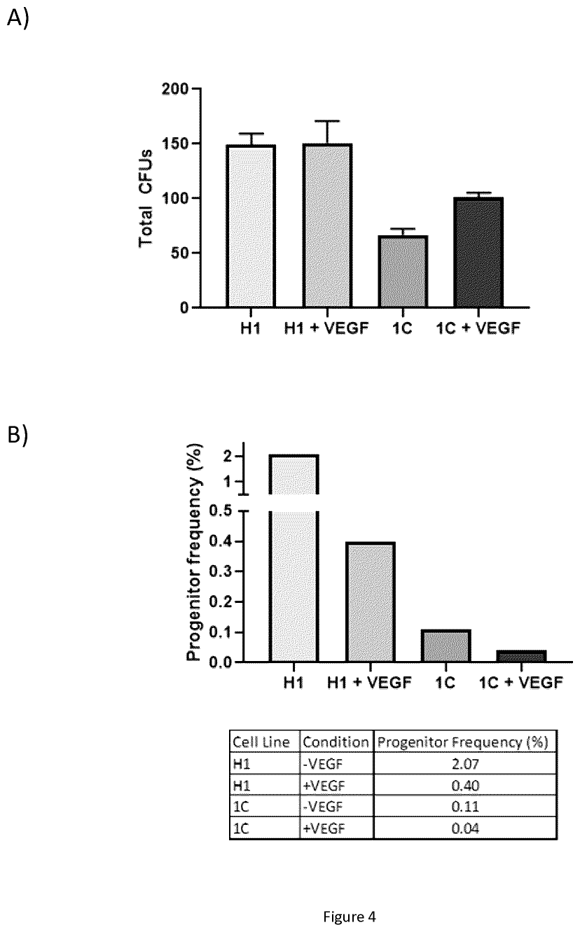

hPSC were differentiated for 12 days usingSTEMdiff™ Hematopoietic—EB reagents either with or without VEGF from days 3-12 (as described above). The output cells were harvested, dissociated, and magnetically enriched for CD34⁺ cells (as described above). The enriched cells were seeded at a density of 1×10⁴ cells per well and cultured in MethoCult™ SF H4636 (STEMCELL Technologies), in accordance with the manufacturer's instructions. After 12 days, images and counts were obtained using the STEM™ Vision instrument (STEMCELL Technologies), in accordance with manufacturer's instructions. FIG. 4A shows total #CFU per 1×10⁴ cells plated, and the enumerated population includes pooled CFU-GM, CFU-GEMM and BFU-E. For H1 cells (ES) and WLS-1C (iPS) the number of CFUs obtained from the CD34⁺ hematopoietic progenitor cells derived without VEGF was similar to those containing VEGF.

Further, the lymphoid potential of the hematopoietic progenitor cells (differentiated as described hereinabove) was assessed in a limiting dilution assay. Briefly, FACS sorted CD34⁺ cells (differentiated and sorted as described herein) were seeded onto plates coated with StemSpan Lymphoid Differentiation Coating Material in StemSpan Lymphoid Expansion Supplement (both, STEMCELL Technologies) at increasing numbers: 10, 30, 100, 300, 1000, 3000 and 5000 cells per well. Twelve replicate wells at each cell number were set-up, and wells containing ≥200 CD7+ cells were scored as positive. The progenitor frequency was determined using the Extreme Limiting Dilution Assay tool with these results. The frequency of hematopoietic progenitors with lymphoid potential derived in the absence of VEGF (during the hematopoietic differentiation stage) was higher in comparison to the condition including VEGF: 2.07 vs 0.4% for H1 (ES cells), and 0.11 vs 0.04% for 1C (iPS cells) (FIG. 4B).

Therefore, removal of VEGF does not appear to negatively impact the generation of CD34⁺ cells with multipotent potential and may in fact increase lymphoid potential.

### Example 7: Deriving Lymphoid Progenitors from Differentiated Hematopoietic Progenitors

In an expanded experiment using enriched CD34⁺ cells (as obtained in accordance with Example 5) differentiated across a greater number of PSC cells lines were tested in downstream differentiation experiments. The enriched CD34⁺ cells were differentiated to lymphoid progenitors using StemSpan™ Lymphoid Progenitor Expansion Medium (StemSpan™ SFEM II supplemented with Lymphoid Progenitor Expansion Supplement (STEMCELL Technologies)). Two hours prior to seeding the cells, non-tissue culture treated cultureware was coated with the StemSpan™ Lymphoid Differentiation Coating Material diluted to 1× in PBS (STEMCELL Technologies) according to the manufacturer's recommendations. After the two-hour incubation at room temperature, the coating material was aspirated and the wells were rinsed with PBS. It is also possible to incubate the plates at 2-8° C. overnight.

Depending on the cell culture plate format, an appropriate number of the enriched CD34⁺ cells of Example 5 were seeded into each well in an appropriate volume of StemSpan™ Lymphoid Progenitor Expansion Medium (STEMCELL Technologies) (see the table below).

| | Volume of StemSpan ™ | | |
|---|---|---|---|
| | Lymphoid Progenitor | | Number of CD34+ cells/ |
| Cultureware | Expansion Medium | | well at each reseeding |
| 96-well plate | 100 | μl/well | 5 × 10³ |
| 24-well plate | 500 | μl/well | 2.5 × 10⁴ |
| 12-well plate | 1 | mL/well | 5 × 10⁴ |
| 6-well plate | 2.5 | mL/well | 1.25 × 10⁵ |

After 3-4 days in culture, an additional volume equal to the original volume of StemSpan™ Lymphoid Progenitor Expansion Medium was added to each well. After another 3-4 days in culture (and for subsequent 3-4 day cycles as appropriate), a half-volume medium change of StemSpan™ Lymphoid Progenitor Expansion Medium was performed taking care to not disrupt the cells.

Approximately 1-week after initially seeding the enriched CD34⁺ cells in StemSpan™ Lymphoid Progenitor Expansion Medium, non-tissue culture treated cultureware was coated with the StemSpan™ Lymphoid Differentiation Coating Material as described above. The cells were then carefully resuspended and transferred into the freshly coated cultureware, taking care to not scrape the bottom of the plate or dislodge any adherent cells. The cells were then incubated at 37° C. for an additional 1 week. Half-media changes were performed every 3-4 days was as described above.

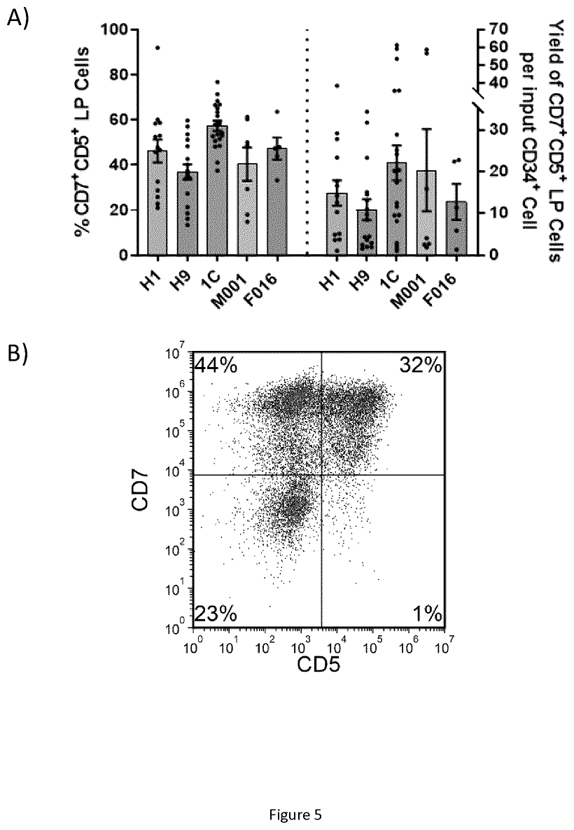

H1, H9, WLS-1C, STiPS-M001, and STiPS-F016 PSC lines efficiently differentiated to CD5⁺CD7⁺ lymphoid progenitor cells via a CD34⁺ hematopoietic progenitor cell intermediate. The mean frequency of CD5⁺CD7⁺ lymphoid progenitor cells, as calculated in accordance with Example 3, across all PSC lines ranged between 37% and 57% (FIG. 5A). The mean yield of CD5⁺CD7⁺ lymphoid progenitor cells per input CD34⁺ hematopoietic progenitor cell, as calculated in accordance with Example 3, across all PSC lines ranged between, 11 and 22 cells (FIG. 5A). Representative flow cytometry plot of thusly-derived CD5⁺CD7⁺ lymphoid progenitor cells is shown in FIG. 5B.

### Example 8: Impacts of Varying the Incubation Period on Hematopoietic Progenitor Cell Differentiation and Downstream Lymphoid Progenitor Cells

The experiments In Example 4 were conducted using a differentiation duration of 10 days. This included a first 3-day period to form a population of early mesoderm cells followed by a subsequent 7-day period to form hematopoietic progenitor cells. A series of experiments were conducted comparing the foregoing 10-day protocol to a longer 12-day protocol, wherein the subsequent 7-day period was extended to 9-days.

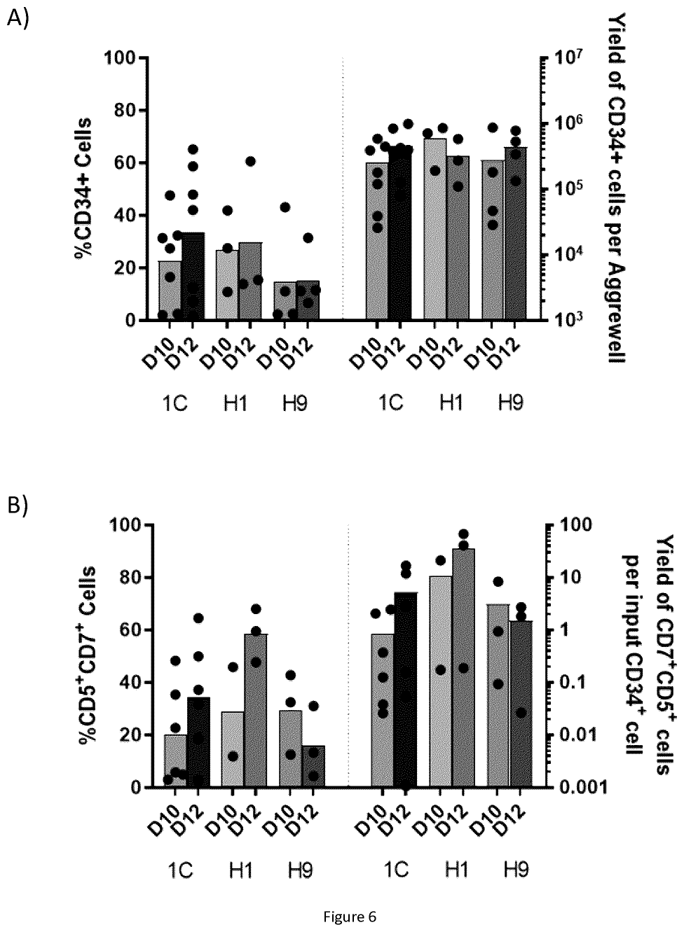

In 2 of 3 PSC lines tested (1C and H1), the longer duration improved not only the frequency and yield per well of a 6-well Aggrewell™ 400 plate of CD34⁺ hematopoietic progenitor cells (FIG. 6A), but also downstream lymphoid potential as measured by frequency and yield of CD5⁺CD7⁺ lymphoid progenitor cells, as generated in accordance with Example 7 (FIG. 6B).

### Example 9: Deriving CD4⁺CD8⁺ T Cells from Lymphoid Progenitors

Two hours prior to the start of this T cell differentiation protocol, non-tissue culture treated cultureware was coated with the StemSpan™ Lymphoid Differentiation Coating Material at 1× as described in Example 7.

The CD5⁺CD7⁺ lymphoid progenitors of Example 7 were seeded into the freshly coated cultureware at 0.5-1×10⁶ cells per mL in StemSpan™ T Cell Progenitor Maturation Medium (StemSpan™ SFEM II supplemented with StemSpan™ T Cell Progenitor Maturation Supplement (STEMCELL Technologies)) and cultured for 2 weeks at 37° C. After the first 3-4 day period, an additional volume equal to the original volume of StemSpan™ T Cell Progenitor Maturation Medium was added to each well. Every 3-4 days thereafter half-volume media changes were performed taking care to not disrupt the cells.

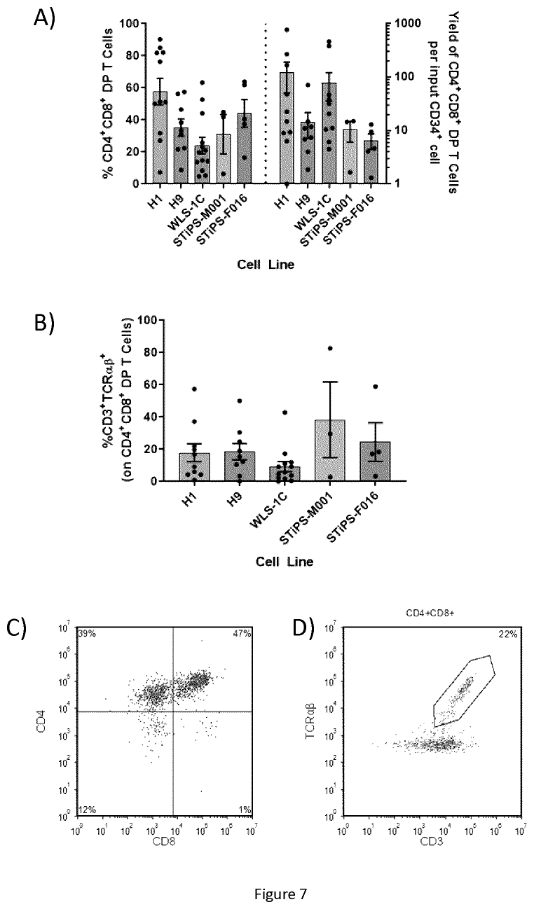

After the 2-week incubation period, the CD4⁺CD8⁺ T cells were harvested and analyzed by flow cytometry, essentially as described in Example 3. FIG. 7A shows the frequency and yield per input CD34⁺ cell of CD4⁺CD8⁺ T cells derived from H1, H9, WLS-1C, STiPS-M001, and STiPS-F016 PSC lines in accordance with Examples 2-5, Example 7, and this Example 9. The mean frequency of CD4⁺CD8⁺ T cells across all PSC lines tested ranged from 24% to 58%. The mean yield of CD4⁺CD8⁺ T cells per input CD34⁺ cell across all PSC lines tested ranged from 7 to 120. FIG. 7B shows the frequency of CD3⁺TCRαβ⁺ cells among the CD4⁺CD8⁺ T cells derived from each PSC line. The mean frequency of CD3⁺TCRαβ⁺ cells among CD4⁺CD8⁺ cells across all PSC lines ranged from 9% to 38%. Representative flow cytometry plots showing expression of CD4 and CD8 are shown for H1-derived CD4⁺CD8⁺ T cells (FIG. 7C) and H1-derived CD3⁺TCRαβ⁺ cells gated on CD4⁺CD8⁺ T cells (FIG. 7D)

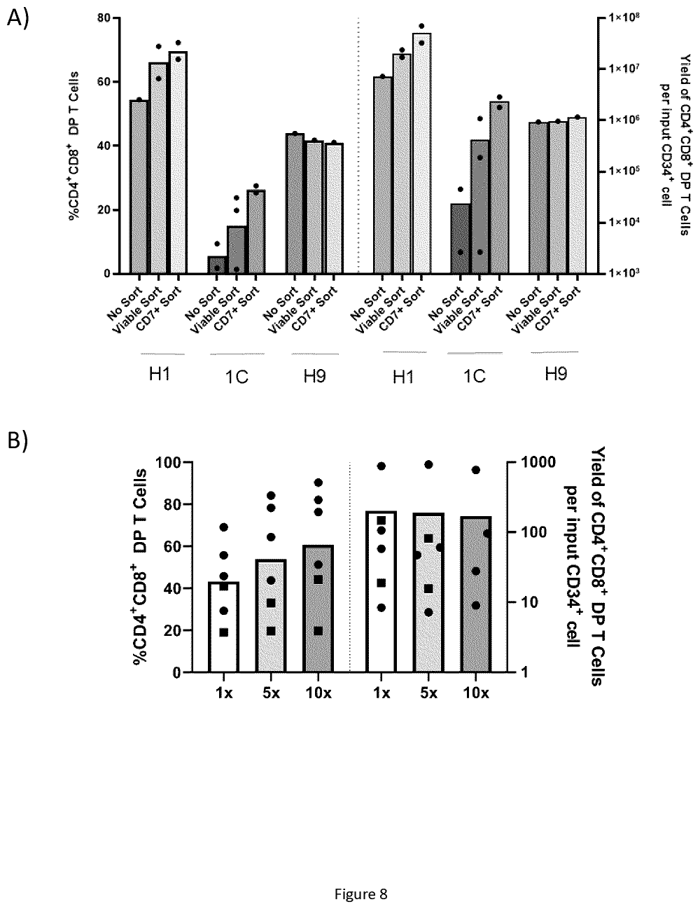

If FACS is available, sorting on viable cells using a combination of FSC vs SSC, and a viability dye such as DRAQ7 or 7-AAD prior to seeding the CD5⁺CD7⁺ lymphoid progenitors improved the frequency and yield per input CD34⁺ cell of CD4⁺CD8⁺ T cells. FIG. 8A shows the effects of differentiating sorted viable cells or CD7⁺ cells on the frequency and yield per input CD34⁺ cell of CD4⁺CD8⁺ T cells derived from H1, WLS-1C, and H9 PSC lines in accordance with Examples 2-5, Example 7, and this Example 9. A higher frequency and yield per input CD34⁺ cell of CD4⁺CD8⁺ T cells was observed for H1 and WLS-1C using seeded viable cells and CD7⁺ cells as compared to seeding unsorted cells of Example 7.

Varying the number of CD5⁺CD7⁺ lymphoid progenitor cells seeded into the freshly coated cultureware improved the frequency and yield per input CD34⁺ cell of CD4⁺CD8⁺ T cells. FIG. 8B shows the effects of increasing the number of seeded CD5⁺CD7⁺ cells on the frequency and yield per input CD34⁺ cell of CD4⁺CD8⁺ T cells derived from H1, WLS-1C, and H9 PSC lines in accordance with Examples 2-5 and Example 7. A higher frequency and yield per input CD34⁺ cell of CD4⁺CD8⁺ T cells was observed for H1 and WLS-1C with increased number of seeded CD5⁺CD7⁺ cells.

### Example 10: Deriving CD4⁺CD8⁺CD3⁺TCRα6⁺ Single Positive T Cells from Double Positive T Cells

Two hours prior to the start of this T cell differentiation protocol, non-tissue culture treated cultureware was coated with the StemSpan™ Lymphoid Differentiation Coating Material at 1× as described in Example 7.

The CD4⁺CD8⁺ T cells of Example 9 were seeded into freshly coated cultureware at 1×10⁶ cells/mL in CD8 SP T Cell Maturation Medium (StemSpan™ SFEM II supplemented with StemSpan™ T Cell Progenitor Maturation Supplement (STEMCELL Technologies) and approximately 10 ng/mL Human Recombinant IL-15 (STEMCELL Technologies)) and including: either ImmunoCult™ Human CD3/CD28/CD2 T Cell Activator (STEMCELL Technologies) or ImmunoCult™ Human CD3/CD28 T Cell Activator (STEMCELL Technologies) at 0.5× the recommended concentration (i.e. 12.5 ug/mL). The cells were cultured for 3-4 days at 37° C. and 5% CO₂. After 3-4 days, the wells were topped up with fresh CD8 SP T Cell Maturation Medium, but not including the ImmunoCult™ T cell activator and returned to incubate at 37° C. and 5% CO₂ for an additional 3-4 days.

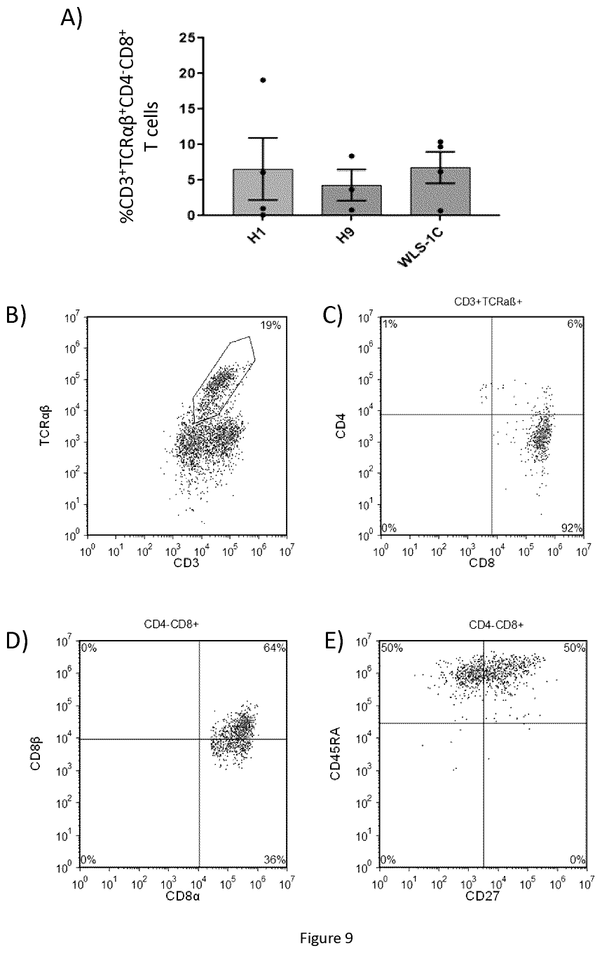

After the additional 3-4 day incubation, single positive CD8⁺ T cells were harvested and analyzed by flow cytometry, essentially as described in Example 3. FIG. 9A shows that CD3⁺TCRαβ+CD4-CD8⁺ single positive T cells could be generated from H1-, H9-, and WLS-1C-derived CD4⁺CD8⁺ T cells (generated in accordance with Example 9). The mean frequency of CD3⁺TCRαβ+CD4-CD8⁺ single positive T cells across all PSC lines tested ranged from 4% to 7%. FIGS. 9B-E further show that the CD3⁺TCRαβ+CD4-CD8⁺ single positive T cells express the CD8αβ heterodimer, rather than the CD8αα homodimer indicative of more immature cells, and the CD45RA and CD27 markers that may be indicative of a mature naïve phenotype.

### Example 11: Deriving NK Cells from Lymphoid Progenitors

The CD5⁺CD7⁺ lymphoid progenitors of Example 7 were seeded into the uncoated cultureware at 1×10⁵ cells per mL in StemSpan™ NK Cell Differentiation Medium (StemSpan™ SFEM II supplemented with StemSpan™ NK Cell Differentiation Supplement (STEMCELL Technologies) and cultured for 2 weeks at 37° C. After the first 3-4 day period, an additional volume equal to the original volume of StemSpan™ NK Cell Differentiation Medium was added to each well. Every 3-4 days thereafter half-volume media changes were performed taking care to not disrupt the cells.

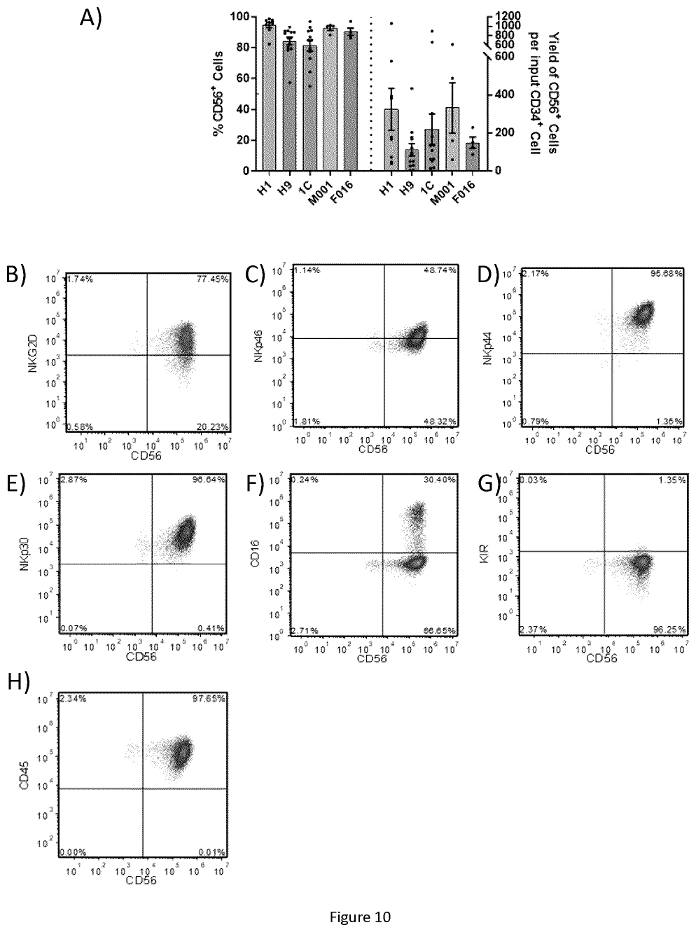

After the 2-week incubation period, the CD56⁺ NK cells were harvested and analyzed by flow cytometry, essentially as described in Example 3. FIG. 10A shows the frequency and yield per input CD34⁺ cell of CD56⁺ NK cells derived from H1, H9, WLS-1C, STiPS-M001, and STiPS-F016 PSC lines in accordance with Examples 2-5, Example 7 and this Example 11. The mean frequency of CD56⁺ NK cells across all PSC lines tested ranged from 81% to 95%. The mean yield of CD56⁺ NK cells per input CD34⁺ cell across all PSC lines tested ranged from 112 to 332. Representative flow cytometry plots showing expression of various NK cell markers are shown for STiPS-M001 PSC (FIG. 10B-H).

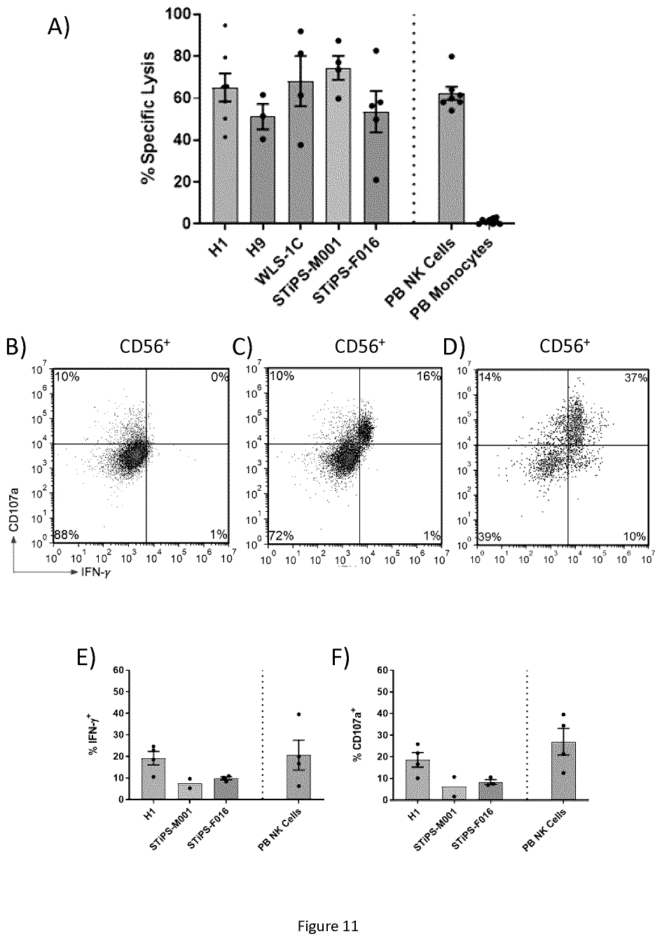

To determine if the PSC-derived NK cells were functionally active, they were tested in a cytotoxicity assay on Calcein-AM (CAM) labeled K562 cells. Peripheral blood (PB) NK cells, isolated using EasySep™ Human NK Cell Isolation Kit (STEMCELL Technologies), and monocytes, isolated using EasySep™ Human Monocyte Isolation Kit (STEMCELLTechnologies) were used as positive and negative controls, respectively. Test and control cells were co-cultured with Calcein AM (CAM)-labeled K562 cells for 4 hours at a 2.5:1 effector (NK) to target cell (K562) ratio. After co-culture, samples of supernatant solution were obtained and released fluorescence from lysed cells was measured using a Spectramax™ microplate reader. The % specific lysis was calculated using the following formula: (test release−spontaneous release)/(max release−spontaneous release)*100%. FIG. 11A shows NK cells derived from various PSC lines as described in Examples 2-5, Example 7, and this Example 11 have comparable killing capacity of K562 cells to PB NK cells.

To confirm the results of the Calcein-AM cytotoxicity assay shown in FIG. 11A, a CD107a degranulation assay was performed to demonstrate the ability of the NK cells to degranulate upon stimulation (as described above) with a 1:1 effector: target ratio of unlabeled K562 target cells. CD107a is a lysosomal-associated membrane protein that will be exposed on the cell surface once NK cells are stimulated and degranulate. As a negative control, unactivated H1-derived NK cells were exposed to monensin, brefeldin A (BFA), and an anti-CD107a fluorescent antibody for 4 hours, but not co-cultured with K562 cells (FIG. 11B). NK cells derived from H1 cells and PB NK cells were activated, FIGS. 11C and 11D respectively, by co-culture with K562 cells at a ratio of 1:1 effector (NK cell) to target (K562) cells in the presence of monensin, brefeldin A (BFA), and an anti-CD107a fluorescent antibody for 4 hours. After the 4-hour incubation, cells were isolated, fixed, permeated, and stained for viability, CD56, CD107a, and IFN-γ. The expression of these markers was assessed using flow cytometry essentially as described in Example 3. In contrast to unactivated H1-derived CD56⁺ NK cells, activated H1-derived CD56⁺ NK cells are capable of degranulation and cytolytic activity similar to that of activated PB CD56⁺ NK cells. These results were extended across STiPS-M001 and STiPS-F016 PSC lines (FIGS. 11E and 11F).

### Example 12: PSC May be Maintained in Various Culture Media without Negatively Impacting Downstream Differentiation

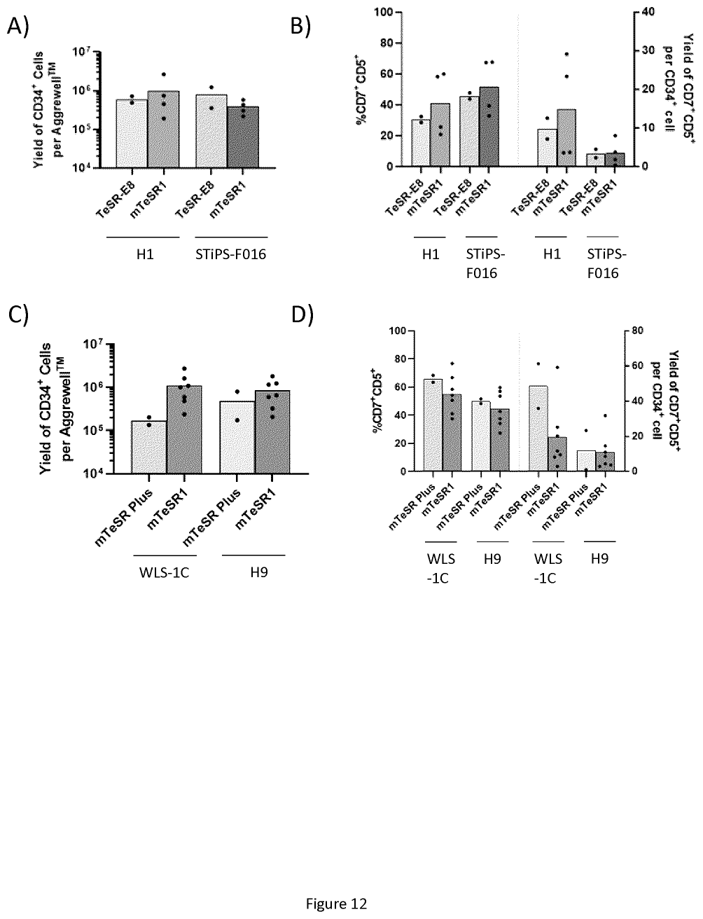

Various PSC lines were maintained in various PSC maintenance media as described in Example 1. The PSC were differentiated and analyzed as outlined Examples 2 through 5, and Example 7. FIG. 12 shows that each of mTeSR™1, TeSR-E8, and mTeSR Plus are compatible with downstream differentiation.

FIG. 12A shows the yield per well of a 6-well Aggrewell™ microwell device of CD34⁺ hematopoietic progenitor cells derived from H1 and STiPS-F016 PSC lines in derivation medium followed by first medium. The mean yield of H1- and STiPS-F016-derived CD34⁺ hematopoietic progenitor cells was comparable when using PSCs maintained in either TeSR-E8 or mTeSR1. FIG. 12B shows the frequency and yield per input CD34⁺ hematopoietic progenitor cell of CD5⁺CD7⁺ lymphoid progenitor cells derived from H1 PSC lines. Both TeSR-E8- and mTeSR1-maintained PSC produced comparable frequencies and yield of CD5⁺CD7⁺ lymphoid progenitor cells.

FIG. 12C shows the yield per well of a 6-well Aggrewell™ microwell device of CD34⁺ hematopoietic progenitor cells derived from WLS-1C and H9 PSC lines in derivation medium followed by first medium. The mean yield of WLS-1C- and H9-derived CD34⁺ hematopoietic progenitor cells was comparable when using PSCs maintained in either mTeSR Plus or mTeSR1. FIG. 12D shows the frequency and yield per input CD34⁺ hematopoietic progenitor cell of CD5⁺CD7⁺ lymphoid progenitor cells derived from WLS-1C and H9 PSC lines. Both mTeSR Plus- and mTeSR1-maintained PSC produced comparable frequencies and yield of CD5⁺CD7⁺ lymphoid progenitor cells.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of differentiating CD34⁺ hematopoietic progenitor cells, comprising culturing a population of early mesoderm cells in a first medium, wherein the first medium comprises a basal medium supplemented with one or more of thrombopoeitin (TPO), stem cell factor (SCF) and FMS-like tyrosine kinase 3 ligand (FLT3L), and excludes any combination of exogenously added agonists selected from the group consisting of an agonist of BMP signaling, an agonist of fibroblast growth factor (FGF) signaling, and an agonist of vascular endothelial growth factor (VEGF) signaling.

2-4. (canceled)

5. The method according to claim 1, wherein the first medium is serum-free.

6. The method according to claim 1, further comprising culturing the population of early mesoderm cells in the absence of exogenously added stroma- and/or feeder-cells.

7. The method according to claim 1, wherein culturing the population of early mesoderm cells in the first medium is for between 3 and 15 days.

8-9. (canceled)

10. The method according to claim 1, further comprising deriving the population of early mesoderm cells from a population of pluripotent stem cells (PSC).

11. The method according to claim 10, further comprising culturing the population of PSC in a derivation medium, wherein the derivation medium comprises a second basal medium supplemented with one or more of the agonist of BMP signaling, the agonist of FGF signaling, and the agonist of VEGF signaling to obtain a differentiated population of PSC-derived early mesoderm cells.

12. The method according to claim 11, wherein the agonist of BMP signaling is a bone morphogenic protein, and optionally the bone morphogenic protein is BMP4.

13. The method according to claim 11, wherein the agonist of FGF signaling is a fibroblast growth factor, and optionally the fibroblast growth factor is FGF2.

14. The method according to claim 11, wherein the agonist of VEGF signaling is a vascular endothelial growth factor.

15. The method according to claim 11, wherein the derivation medium is serum-free.

16. The method according to claim 11, further comprising culturing the PSC in the absence of exogenously added stroma- and/or feeder-cells.

17. The method according to claim 11, wherein culturing the PSC in the derivation medium is for between 1 and 7 days.

18. The method according to any one of claim 11 to 17, qfurther comprising forming the PSC into aggregates in the derivation medium or prior to culturing the PSC in the derivation medium.

19. (canceled)

20. The method according to claim 18 or 19, wherein the aggregates are formed in a microwell device.

21. The method according to claim 1, further comprising obtaining a multi-potent lymphoid progenitor cells after culturing the differentiated hematopoietic progenitor cells in a lymphoid differentiation medium under serum-free conditions and/or in the absence of exogenously added stroma- and/or feeder cells.

22-24. (canceled)

25. A medium for differentiating hematopoietic progenitor cells from a population of early mesoderm cells, the media comprising a basal medium supplemented with one or more of thrombopoeitin (TPO), stem cell factor (SCF) and FMS-like tyrosine kinase 3 ligand (FLT3L), and excludes any combination of exogenously added agonists selected from the list consisting of an agonist of BMP signaling, an agonist of fibroblast growth factor (FGF) signaling, and an agonist of vascular endothelial growth factor (VEGF) signaling.

26-27. (canceled)

28. The medium according to claim 25, wherein the medium is serum-free.

29. (canceled)

30. The medium according to claim 25, wherein the agonist of fibroblast growth factor (FGF) signaling is a fibroblast growth factor, and optionally FGF2.

31. The medium according to claim 25, wherein the agonist of BMP signaling is a bone morphogenic protein, and optionally BMP4.

32. The medium according to claim 25, wherein the agonist of vascular endothelial growth factor (VEGF) signaling is VEGF.

33. (canceled)
